SEQUENCE LISTING (63 sequences)

SEQ ID NO: 1 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDIGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPHSAIGLEDNASAAALLGLRSWQTRALLIKALIDQGYADQILVSNDWLFGFSSYVTNIMDVMDRVNPDGMAFIPLRVIPFLREKGVPDETLETIMVDNPARFLSPTLRAS

SEQ ID NO: 2 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDIGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPHSAIGLEDNASAAAILGLRSWQTRALLIKALIDQGYADQILVSNDWTFGISSYVTNIMDVLDRVNPDGMAFIPLRVIPFLREKGVPDETLETIMVDNPARFLSPTLRAS

SEQ ID NO: 3 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDIGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPHSAIGLEDNASAAARLGLRSWQTRALLIKALIDQGYADQILVSNDWTFGISSYVTNIMDVLDRVNPDGMAFIPLRVIPFLREKGVPDETLETIMVDNPARFLSPTLRAS

SEQ ID NO: 4 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDIGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPYSAIGLEDNASAAALLGLRSWQTRALLIKALIDQGYADQILVSNDWTFGISSYVTNIMDVMDRVNPDGMAFIPLRVIPFLREKGVPDETLETIMVDNPARFLSPTLRAS

SEQ ID NO: 5 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDIGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPYSAIGLEDNASAAAILGLRSWQTRALLIKALIDQGYADQILVSNDWTFGFSSYVTNIMDVLDRVNPDGMAFIPLRVIPFLREKGVPDETLETIMVDNPARFLSPTLRAS

SEQ ID NO: 6 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDIGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPWSAIGLEDNASAAALLGLRSWQTRALLIKALIDQGYADQILVSNDWTFGFSSYVTNIMDVLDRVNPDGMAFIPLRVIPFLREKGVPDETLETIMVDNPARFLSPTLRAS

SEQ ID NO: 7 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDIGRDVELLAEVSEAADVHIVAATGLWLDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPWSAIGLEDNASAAALLGLRSWQTRALLIKALIDQGYADQILVSNDWTFGISSYVTNIMDVLDRVNPDGMAFIPLRVIPFLREKGVPDETLETIMVDNPARFLSPTLRAS

SEQ ID NO: 8 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDCGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPHSAIGLEDNASAAARLGLRSWQTRALLIKALIDQGYADQILVSNDWLFGFSSYVTNIMDVLDRVNPDGMAFIPLRVIPFLREKGVPDETLETIMVDNPARFLSPTLRAS

SEQ ID NO: 9 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDCGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPWSAIGLEDNASAAALLGLRSWQTRALLIKALIDQGYADQILVSNDWTFGFSSYVTNIMDVLDRVNPDGMAFIPLRVIPFLREKGVPDETLETIMVDNPARFLSPTLRAS

SEQ ID NO: 10 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDCGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPWSAIGLEDNASAAARLGLRSWQTRALLIKALIDQGYADQILVSNDWLFGFSSYVTNIMDVMDRVNPDGMAFIPLRVIPFLREKGVPDETLETIMVDNPARFLSPTLRAS

SEQ ID NO: 11 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDCGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPYSAIGLEDNASAAAILGLRSWQTRALLIKALIDQGYADQILVSNDWLFGFSSYVTNIMDVMDRVNPDGMAFIPLRVIPFLREKGVPDETLETIMVDNPARFLSPTLRAS

SEQ ID NO: 12 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDCGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPHSAIGLEDNASAAALLGLRSWQTRALLIKALIDQGYADQILVSNDWTFGISSYVTNIMDVLDRVNPDGMAFIPLRVIPFLREKGVPDETLETIMVDNPARFLSPTLRAS

SEQ ID NO: 13 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDCGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPHSAIGLEDNASAAAILGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 14 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDCGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPHSAIGLEDNASAAALLGLRSWQTRALLIKALIDQGYADQILVSNDWLFGFSSYVTNIMDVLDRVNPDGMAFIPLRVIPFLREKGVPDETLETIMVDNPARFLSPTLRAS

SEQ ID NO: 15 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDCGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPHSAIGLEDNASAAARLGLRSWQTRALLIKALIDQGYADQILVSNDWTFGISSYVTNIMDVMDRVNPDGMAFIPLRVIPFLREKGVPDETLETIMVDNPARFLSPTLRAS

SEQ ID NO: 16 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDCGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPWSAIGLEDNASAAALLGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 17 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDHGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPHSAIGLEDNASAAARLGLRSWQTRALLIKALIDQGYADQILVSNDWTFGISSYVTNIMDVLDRVNPDGMAFIPLRVIPFLREKGVPDETLETIMVDNPARFLSPTLRAS

SEQ ID NO: 18 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDHGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPYSAIGLEDNASAAALLGLRSWQTRALLIKALIDQGYADQILVSNDWTFGISSYVTNIMDVLDRVNPDGMAFIPLRVIPFLREKGVPDETLETIMVDNPARFLSPTLRAS

SEQ ID NO: 19 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDHGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPHSAIGLEDNASAAAILGLRSWQTRALLIKALIDQGYADQILVSNDWLFGFSSYVTNIMDVMDRVNPDGMAFIPLRVIPFLREKGVPDETLETIMVDNPARFLSPTLRAS

SEQ ID NO: 20 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDHGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPWSAIGLEDNASAAAILGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 21 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDHGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPHSAIGLEDNASAAALLGLRSWQTRALLIKALIDQGYADQILVSNDWTFGISSYVTNIMDVLDRVNPDGMAFIPLRVIPFLREKGVPDETLETIMVDNPARFLSPTLRAS

SEQ ID NO: 22 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDHGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPWSAIGLEDNASAAALLGLRSWQTRALLIKALIDQGYADQILVSNDWTFGISSYVTNIMDVMDRVNPDGMAFIPLRVIPFLREKGVPDETLETIMVDNPARFLSPTLRAS

SEQ ID NO: 23 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPHSAIGLEDNASAAALLGLRSWQTRALLIKALIDQGYADQILVSNDWTFGISSYVTNIMDVLDRVNPDGMAFIPLRVIPFLREKGVPDETLETIMVDNPARFLSPTLRAS

SEQ ID NO: 24 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPHSAIGLEDNASAAARLGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 25 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPWSAIGLEDNASAAAILGLRSWQTRALLIKALIDQGYADQILVSNDWLFGFSSYVTNIMDVLDRVNPDGMAFIPLRVIPFLREKGVPDETLETIMVDNPARFLSPTLRAS

SEQ ID NO: 26 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPWSAIGLEDNASAAAILGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 27 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPYSAIGLEDNASAAARLGLRSWQTRALLIKALIDQGYADQILVSNDWLFGISSYVTNIMDVLDRVNPDGMAFIPLRVIPFLREKGVPDETLETIMVDNPARFLSPTLRAS

SEQ ID NO: 28 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPHSAIGLEDNASAAALLGLRSWQTRALLIKALIDQGYADQILVSNDWLFGFSSYVTNIMDVLDRVNPDGMAFIPLRVIPFLREKGVPDETLETIMVDNPARFLSPTLRAS

SEQ ID NO: 29 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPWSAIGLEDNASAAALLGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 30 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPYSAIGLEDNASAAAILGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 31 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPHSAIGLEDNASAAAILGLRSWQTRALLIKALIDQGYADQILVSNDWLFGISSYVTNIMDVLDRVNPDGMAFIPLRVIPFLREKGVPDETLETIMVDNPARFLSPTLRAS

SEQ ID NO: 32 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPHSAIGLEDNASAAAILGLRSWQTRALLIKALIDQGYADQILVSNDWTFGISSYVTNIMDVMDRVNPDGMAFIPLRVIPFLREKGVPDETLETIMVDNPARFLSPTLRAS

SEQ ID NO: 33 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPWSAIGLEDNASAAARLGLRSWQTRALLIKALIDQGYADQILVSNDWLFGFSSYVTNIMDVMDRVNPDGMAFIPLRVIPFLREKGVPDETLETIMVDNPARFLSPTLRAS

SEQ ID NO: 34 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPYSAIGLEDNASAAALLGLRSWQTRALLIKALIDQGYADQILVSNDWLFGFSSYVTNIMDVLDRVNPDGMAFIPLRVIPFLREKGVPDETLETIMVDNPARFLSPTLRAS

SEQ ID NO: 35 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPYSAIGLEDNASAAALLGLRSWQTRALLIKALIDQGYADQILVSNDWLFGISSYVTNIMDVMDRVNPDGMAFIPLRVIPFLREKGVPDETLETIMVDNPARFLSPTLRAS

SEQ ID NO: 36 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWLDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPWSAIGLEDNASAAALLGLRSWQTRALLIKALIDQGYADQILVSNDWLFGFSSYVTNIMDVMDRVNPDGMAFIPLRVIPFLREKGVPDETLETIMVDNPARFLSPTLRAS

SEQ ID NO: 37 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWLDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPWSAIGLEDNASAAALLGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 38 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPHSAIGLEDNASAAALLGLRSWQTRALLIKALIDQGYADQILVSNDWLFGISSYVTNIMDVLDRVNPDGMAFIPLRVIPFLREKGVPDETLETIMVDNPARFLSPTLRAS

SEQ ID NO: 39 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPHSAIGLEDNASAAARLGLRSWQTRALLIKALIDQGYADQILVSNDWTFGFSSYVTNIMDVLDRVNPDGMAFIPLRVIPFLREKGVPDETLETIMVDNPARFLSPTLRAS

SEQ ID NO: 40 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPHSAIGLEDNASAAARLGLRSWQTRALLIKALIDQGYADQILVSNDWTFGISSYVTNIMDVMDRVNPDGMAFIPLRVIPFLREKGVPDETLETIMVDNPARFLSPTLRAS

SEQ ID NO: 41 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPWSAIGLEDNASAAALLGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 42 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPYSAIGLEDNASAAALLGLRSWQTRALLIKALIDQGYADQILVSNDWLFGFSSYVTNIMDVLDRVNPDGMAFIPLRVIPFLREKGVPDETLETIMVDNPARFLSPTLRAS

SEQ ID NO: 43 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPHSAIGLEDNASAAALLGLRSWQTRALLIKALIDQGYADQILVSNDWTFGISSYVTNIMDVMDRVNPDGMAFIPLRVIPFLREKGVPDETLETIMVDNPARFLSPTLRAS

SEQ ID NO: 44 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPWSAIGLEDNASAAALLGLRSWQTRALLIKALIDQGYADQILVSNDWLFGFSSYVTNIMDVMDRVNPDGMAFIPLRVIPFLREKGVPDETLETIMVDNPARFLSPTLRAS

SEQ ID NO: 45 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPHSAIGLEDNASAAALLGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 46 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPHSAIGLEDNASAAARLGLRSWQTRALLIKALIDQGYADQILVSNDWLFGFSSYVTNIMDVLDRVNPDGMAFIPLRVIPFLREKGVPDETLETIMVDNPARFLSPTLRAS

SEQ ID NO: 47 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPWSAIGLEDNASAAALLGLRSWQTRALLIKALIDQGYADQILVSNDWLFGFSSYVTNIMDVLDRVNPDGMAFIPLRVIPFLREKGVPDETLETIMVDNPARFLSPTLRAS

SEQ ID NO: 48 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWLDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPHSAIGLEDNASAAALLGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 49 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWLDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDHIPWSAIGLEDNASAAALLGLRSWQTRALLIKALIDQGYADQILVSNDWTFGFSSYVTNIMDVLDRVNPDGMAFIPLRVIPFLREKGVPDETLETIMVDNPARFLSPTLRAS

SEQ ID NO: 50 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWLDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDRIPWSAIGLEDNASAAALLGLRSWQTRALLIKALIDQGYADQILVSNDWLFGFSSYVTNIMDVMDRVNPDGMAFIPLRVIPFLREKGVPDETLETIMVDNPARFLSPTLRAS

SEQ ID NO: 51 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPWSAIGLEDNASAAALLGLRSWQTRALLIKALIDQGYADQILVSNDWTFGISSYVTNIMDVLDRVNPDGMAFIPLRVIPFLREKGVPDETLETIMVDNPARFLSPTLRAS

SEQ ID NO: 52 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDLGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPWSAIGLEDNASAAALLGLRSWQTRALLIKALIDQGYADQILVSNDWTFGISSYVTNIMDVMDRVNPDGMAFIPLRVIPFLREKGVPDETLETIMVDNPARFLSPTLRAS

SEQ ID NO: 53 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDIGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPHSAIGLEDNASAAALLGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 54 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDIGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPWSAIGLEDNASAAALLGLRSWQTRALLIKALIDQGYADQILVSNDWLFGFSSYVTNIMDVMDRVNPDGMAFIPLRVIPFLREKGVPDETLETIMVDNPARFLSPTLRAS

SEQ ID NO: 55 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDIGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPWSAIGLEDNASAAALLGLRSWQTRALLIKALIDQGYADQILVSNDWTFGFSSYVTNIMDVLDRVNPDGMAFIPLRVIPFLREKGVPDETLETIMVDNPARFLSPTLRAS

SEQ ID NO: 56 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDIGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPWSAIGLEDNASAAALLGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 57 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDIGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPWSAIGLEDNASAAALLGLRSWQTRALLIKALIDQGYADQILVSNDWTFGISSYVTNIMDVMDRVNPDGMAFIPLRVIPFLREKGVPDETLETIMVDNPARFLSPTLRAS

SEQ ID NO: 58 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPHSAIGLEDNASAAALLGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 59 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPHSAIGLEDNASAAALLGLRSWQTRALLIKALIDQGYADQILVSNDWTFGISSYVTNIMDVLDRVNPDGMAFIPLRVIPFLREKGVPDETLETIMVDNPARFLSPTLRAS

SEQ ID NO: 60 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPWSAIGLEDNASAAALLGLRSWQTRALLIKALIDQGYADQILVSNDWLFGISSYVTNIMDVLDRVNPDGMAFIPLRVIPFLREKGVPDETLETIMVDNPARFLSPTLRAS

SEQ ID NO: 61 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPWSAIGLEDNASAAALLGLRSWQTRALLIKALIDQGYADQILVSNDWTFGFSSYVTNIMDVLDRVNPDGMAFIPLRVIPFLREKGVPDETLETIMVDNPARFLSPTLRAS

SEQ ID NO: 62 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPWSAIGLEDNASAAALLGLRSWQTRALLIKALIDQGYADQILVSNDWTFGFSSYVTNIMDVMDRVNPDGMAFIPLRVIPFLREKGVPDETLETIMVDNPARFLSPTLRAS

SEQ ID NO: 63 - protein, 336 aa, Artificial sequence
  PTE Variant amino acid sequence
ITNSGDRINTVRGPITISEAGFTLMHEHICGSSAGFLRAWPEFFGSRDALAEKAVRGLRRARAAGVRTIVDVSTFDMGRDVELLAEVSEAADVHIVAATGLWFDPPLSMRLRSVEELTQFFLREIQYGIEDTGIRAGIIKVATTGKATPFQERVLRAAARASLATGVPVTTHTDASQRDGEQQADIFESEGLDPSRVCIGHSDDTDDLDYLTALAARGYLIGLDGIPWSAIGLEDNASAAALLGLRSWQTRALLIKALIDQGYADQILVSNDWTFGISSYVTNIMDVMDRVNPDGMAFIPLRVIPFLREKGVPDETLETIMVDNPARFLSPTLRAS

(19) United States
(12) Patent Application Publication
(10) Pub. No.: US 2021/0178207 A1
(43) Pub. Date: Jun. 17, 2021

(54) DESIGNED, EFFICIENT AND BROAD-SPECIFICITY ORGANOPHOSPHATE HYDROLASES

(71) Applicant: Yeda Research and Development Co. Ltd., Rehovot (IL)

(72) Inventors: Sarel FLEISHMAN, Rehovot (IL); Dan S. TAWFIK, Rehovot (IL); Olga KHERSONSKY, Rehovot (IL)

(73) Assignee: Yeda Research and Development Co. Ltd., Rehovot (IL)

(21) Appl. No.: 17/267,816

(22) PCT Filed: Aug. 14, 2019
(86) PCT No.: PCT/IL2019/050916

§ 371 Date: Feb. 11, 2021

(30) Foreign Application Priority Data
Aug. 14, 2018 (IL) 261157

(51) Int. Cl. A62D 3/02

(52) U.S. Cl. CPC A62D 3/02; A62D 2101/02

(57) ABSTRACT

Provided herein is a library of designed phosphotriesterase (PTE) enzymes, exhibiting an improved catalytic hydrolysis activity of various substrates, including nerve agents, and a general method of generating and using the same.

### RELATED APPLICATION

This application claims the benefit of priority of Israeli Patent Application No. 261157 filed 14 Aug. 2018, the contents of which are incorporated herein by reference in their entirety.

### SEQUENCE LISTING STATEMENT

The ASCII file, entitled 78359 Sequence Listing.txt, created on 14 Aug. 2019, comprising 188,416 bytes, submitted concurrently with the filing of this application is incorporated herein by reference.

### FIELD AND BACKGROUND OF THE INVENTION

The present invention, in some embodiments thereof, relates to enzymology, and more particularly, but not exclusively, to phosphotriesterase variants designed by a designated computational method to exhibit catalytic activity towards a broad range of organophosphates and chemical warfare nerve agents.

At present, both prophylaxis and post-intoxication treatments of chemical warfare nerve agent (CWNA) poisoning are based on drugs selected to counteract the symptoms caused by accumulation of acetylcholine in cholinergic neurons. Current antidotal regimes consist of pretreatment with pyridostigmine, and of post-exposure therapy that involves administration of a cocktail containing atropine, an oxime reactivator and an anticonvulsant drug such as diazepam. The multi-drug approach against CWNA toxicity has been adopted by many countries and integrated into their civil and military medical protocols. However, it is commonly recognized that these drug regimens suffer from several disadvantages that call for new therapeutic strategies. The preferred approach is to rapidly detoxify the CWNA in the blood before it has had the chance to reach its physiological targets. One way of achieving this objective is by the use of bioscavengers. However, use of the best stoichiometric bioscavenger currently available (human butyrylcholinesterase, hBChE) requires administration of hundreds of milligrams of protein to confer protection against toxic doses of CWNA.

A safer and more effective treatment strategy can be achieved by using a catalytic bioscavenger to rapidly degrade the intoxicating organophosphate (OP) in the circulation. The promiscuous nerve-agent hydrolyzing activities of the enzyme phosphotriesterase (PTE) make it a prime candidate both for prophylactic and post exposure treatment of nerve-agent intoxications. However, efficient in-vivo detoxification using low doses of enzymes (≤50 mg/70 kg) following exposure to toxic doses of nerve agents, requires that the catalytic efficiencies (k_{cat}/K_{M}) of wild-type PTE towards the toxic nerve agent isomers will be increased.

PTE variants that can efficiently hydrolyze V-type nerve agents were disclosed previously [Cherney, I. et al., ACS Chem Biol, 2013, 8(11), pp. 2394-2403]. In-vivo post-exposure activity of one of these variants (C23) was demonstrated in guinea-pigs intoxicated with a lethal dose of VX [Worek, F. et al., Toxicol Lett, 2014, 231(1), pp. 45-54].

Additional background art pertaining to PTE variants includes U.S. Pat. No. 8,735,124, WO2016/092555, WO2018/087759 and Roodveldt, C. and Tawfik, D.S., Protein Eng Des Sel., 2005, 18(1), pp. 51-8. Mutations that alter enzyme activity profiles are essential for adaptation to an organism's changing needs, such as metabolizing new substrates. Such mutations are also highly desired in basic research, biotechnology, and biomedicine to enable efficient and environmentally safe solutions, for instance in the synthesis of useful molecules or the degradation of harmful ones. Most mutations, however, are deleterious to protein activity and stability, constraining the emergence of improved variants through natural evolution or protein engineering. Furthermore, due to mutational epistasis, a mutation's effect on activity depends on whether or not other mutations were previously acquired. In the extreme case, known as sign epistasis, two mutations that are individually deleterious, enhance activity when combined, or vice versa. In natural evolution, mutations usually occur one at a time, and thus, epistatic combinations of mutations must accumulate in a specific order, since all intermediates must be at least as active as their predecessors or they would be purged by selection. The high prevalence of sign epistasis in improved mutants further reduces the likelihood of obtaining beneficial combinations. Protein evolution is additionally constrained by stability-threshold effects, whereby activity-enhancing mutations may destabilize the protein, and therefore accumulate only up to a threshold in which additional mutations are no longer tolerated. To overcome stability-threshold effects, stabilizing mutations, both in proximity to the active-site pocket and in distant regions, are essential for the accumulation of function-enhancing mutations.

Due to epistasis and stability-threshold effects, the evolution of variants with significant enhancement in an enzyme activity demands multiple mutations of different type and affecting different regions of the protein. Laboratory-evolution experiments, for instance, may comprise more than a dozen rounds of genetic diversification and selection for improved mutants, and substantial improvements by three orders of magnitude or more require on average ten mutations. The majority of these mutations occur outside the catalytic pocket and are likely to affect activity only indirectly by enhancing tolerance to function-enhancing mutations. Another complication is that laboratory-evolution experiments are laborious and demand high-throughput or even ultrahigh-throughput screening (>10⁶ variants per round). Such screens, however, are only applicable to certain enzyme activities and typically employ synthetic model substrates.

In principle, computational protein design strategies could bypass the need for multiple rounds of experimental optimization, since they are unconstrained by mutational trajectories. Previous applications of protein design computed favorable point mutants or focused libraries for experimental screening, yielding limited gains in activity, and de novo designed enzymes exhibited low catalytic efficiencies. Overall, computational enzyme design remains a specialized expertise, and still depends on laboratory evolution to reach comparable efficiencies to those seen in natural enzymes. Thus, substantial gaps remain in the understanding and control of the basic principles of enzyme design.

Additional background art pertaining to computational design of protein variants includes U.S. Patent Application Publication No. 2017/0032079, International Patent Application No. WO 2017/017673, Fleishman, S. L. et al., PLoS One, 2011, 6(6), and Goldenzweig, A. et al. Mol Cell., 2016, 63(2), pp. 337-346.

### SUMMARY OF THE INVENTION

Substantial improvements in enzyme activity demand multiple mutations at spatially proximal positions in the active site. Such mutations, however, often exhibit unpredictable epistatic (non-additive) effects on activity. Here, the present invention provides an automated method for designing multipoint mutations at enzyme active sites using phylogenetic analysis and Rosetta design calculations, referred to herein as FuncLib. FuncLib is demonstrated herein using phosphotriesterase; the designed variants of PTE were all active, and most showed activity profiles that significantly differed from the wild type and from one another. Several dozen designs with only 3-6 active-site mutations exhibited 10-4,000-fold higher efficiencies with a range of alternative substrates, including the hydrolysis of the toxic organophosphate nerve agents soman and cyclosarin. FuncLib has also been implemented as a web-server (www(dot)funclib(dot)weizmann(dot)ac(dot)il); it circumvents iterative, high-throughput screens and opens the way to design highly efficient and diverse catalytic repertoires.

Thus, according to an aspect of some embodiments of the present invention, there is provided a protein having a sequence selected from the group consisting of any combination of at least 2 amino acid substitutions of a sequence space afforded for phosphotriesterase (PTE) from Pseudomonas diminuta as an original protein, and listed in Table A:

TABLE A
Position (numbering according to PDB entry: 1HZY

| 106 | 132 | 254 | 257 | 271 | 303 | 306 | 317 |
|---|---|---|---|---|---|---|---|
| C/H/L/M | L | G/R | Y/W | I/R | T | I | L |

In some embodiments, the protein is a hybrid protein wherein the combination of amino acid substitutions is implemented on a PTE protein other than the original protein.

In some embodiments, the protein is characterized by a sequence selected from the group consisting of presented in Table A set forth hereinbelow.

In some embodiments, the protein is characterized by a sequence selected from the group consisting of PTE_28 (SEQ ID NO: 28), PTE_29 (SEQ ID NO: 29), PTE_56 (SEQ ID NO: 56), and PTE_57 (SEQ ID NO: 57).

According to an aspect of some embodiments of the present invention, there is provided a method of detoxification and decontamination of organophosphate agents, which is effected by contacting an area suspected of being contaminated with the organophosphate agents with at least one of the PTE variant proteins provided herein according to some embodiments of the present invention.

In some embodiments, the area is selected from the group consisting of a floor, a wall, a building or a part thereof, a vehicle, a piece of clothing, a piece of equipment, a plant, an animal, and an inanimate object.

In some embodiments, the organophosphate agents are selected from the group consisting of a G-type nerve agent, a V-type nerve agent, and a GV-type nerve agent.

According to an aspect of some embodiments of the present invention, there is provided a method generating a library of enzyme variants (designs), having a diverse improved catalytic activity compared to an original enzyme, the method is effected by:

identifying a group of substitutable residues (substitutable positions) in a first shell and a second shell of an active site of the enzyme, and a group of fixed residues (fixed positions) in these shells;

permuting mutations of the substitutable residues according to a PSSM scoring regimen using a computational software that calculates stability parameters and ranks the permutated mutants according to their energy value, thereby obtaining a stability score list of enzyme variants;

enumerating the enzyme variants resulting from the previous step;

selecting a number of the resulting variants (permutated mutants) at the top of the stability score list, which have at least two mutations in the substitutable residues compared to the original enzyme; and

cloning and expressing that number of variants having top stability score and at least two mutations relative to the original enzyme.

In some embodiments, the method of generating a library of enzyme variants, further includes, prior to identifying substitutable and fixed residues, providing a stabilized variant of the wild-type enzyme using any design-for-stability method (such as PROSS), and using this variant as the original enzyme.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the Drawings:

FIGS. 1A-D illustrate key steps in the computational design method, used to produce a functional phosphotriesterase enzyme repertoire, starting from the structure of bacterial PTE (PDB entry: 1HZY) and the sequence of a stabilized variant or PTE, dPTE2 (SEQ ID NO: 1), wherein FIG. 1A presents the step in which active-site positions are selected for design, and at each position, sequence space is constrained by evolutionary-conservation analysis (PSSM) and mutational-scanning calculations (ΔΔG), FIG. 1B presents the step in which multipoint mutants are exhaustively enumerated using Rosetta atomistic design calculations, FIG. 1C presents the step in which the designs are ranked by energy, and FIG. 1D presents the step wherein the sequences are clustered to obtain a repertoire of diverse, low-energy (namely stable and preorganized) designs for experimental testing, whereas designed positions are colored consistently in all panels;

FIGS. 2A-C present some of the results of the use of the method, according to embodiments of the present invention, FuncLib, in which designed repertoire of phosphotriesterases (PTE) exhibits orders of magnitude improvement in a range of promiscuous activities (numbers in X-axis of FIG. 2B and numbers in Y-axis in FIG. 2C represent the variant number (PTE_X) and the SEQ ID NO: X);

FIG. 3 presents a diagram showing that the designed mutations in the PTE variants provided herein, according to some embodiments of the present invention, exhibit sign-epistatic relationships, wherein each circle represents a mutant of dPTE2 (SEQ ID NO: 1), the area of each circle is proportional to the variant's specific activity in hydrolyzing the aryl ester 2-naphthyl acetate (2NA), and wherein the PROSS designed and stabilized sequence dPTE2 (SEQ ID NO: 1), which was used as the starting point in the method provided herein, exhibits low specific activity, and each of the point mutants exhibits improved specific activity, the specific activity declines in the double mutants, and the quad-mutant, design PTE_6 (SEQ ID NO: 6), substantially improves specific activity relative to all single or double mutants; and

FIG. 4 presents an illustration of the stereochemical properties of the designed active-site pockets that underlie selectivity changes in PTE variants, provided herein according to some embodiments of the present invention, wherein PTE_28 (SEQ ID NO: 28; denoted 28 in FIG. 4) and PTE_29 (SEQ ID NO: 29; denoted 29 in FIG. 4) exhibit a larger active-site pocket than dPTE2 (SEQ ID NO: 1; denoted 1 in FIG. 4) and high catalytic efficiency against bulky V- and G-type nerve agents (in clockwise order from top-left, molecular renderings are based on PDB entries: 1HZY, 6GBJ, 6GBK, and 6GBL; spheres indicate ions of the bimetal center.

### DESCRIPTION OF SPECIFIC EMBODIMENTS OF THE INVENTION

The present invention, in some embodiments thereof, relates to enzymology, and more particularly, but not exclusively, to phosphotriesterase variants designed by a designated computational method to exhibit catalytic activity towards a broad range of organophosphates and chemical warfare nerve agents.

Before explaining at least one embodiment of the invention in detail, it is to be understood that the invention is not necessarily limited in its application to the details of calculation, enumeration and the values of the computational parameters and/or laboratory methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

A method for designing functionally diverse repertoires of an enzyme:

To address the gaps still plaguing contemporary protein design approaches, as discussed in the introductory section hereinabove, the present inventors have developed a protein design strategy that affords sequences of proteins having stable networks of interacting residues at the active site and selects a small set of diverse designs amenable to low-throughput screening. This design paradigm and practical strategy, and the corresponding computational tools and methods provided herein, addresses epistasis by designing dense and pre-organized networks of interacting active-site multipoint mutants. Optionally, the protein design strategy may further include the use of PROSS that addresses stability-threshold effects, by first designing a stable enzyme scaffold. The method does not a priori target a specific substrate, as this demands accurate models of the enzyme transition-state complex, and such models are rarely attainable and are mostly approximate. Rather, the method (design strategy) provided herein, according to some embodiments of the present invention, results in a repertoire of stable and highly efficient proteins (e.g., enzymes, antibodies etc.) that can be screened for the activities of interest.

As presented herein, starting from exemplary enzymes for demonstrative purpose, the method provided herein was used to design functionally diverse repertoires comprising dozens of enzymes that exhibited 10-4,000 fold improvements in a range of activities. The robustness and effectiveness of the herein-presented strategy, can be combined with the previously provided method, implemented publicly available protein-stabilization platform “PROSS” (see, U.S. Patent Application Publication No. 2017/0032079 and WO 2017/017673, each of which is incorporated herein by reference as if fully set forth herein; and e.g., www(dot)pross(dot)weizmann(dot)ac(dot)il/). The method, provided herewith and referred to as “FuncLib” or “AbLift”, has also been implemented as an automated web-accessible server.

Main differences between PROSS, and the method provided herein and implemented in FuncLib and AbLift, is that PROSS designs the protein outside the active/binding site, while FuncLib and AbLift designs the active/binding sites, since PROSS's objective is to stabilise the protein, without changing its structure-related activity. This distinction is of paramount importance: Since there are many positions in any protein open to design of stable variants (>90% of the protein is not directly related to function), PROSS looks only for the safest combinations of mutations, using a combinatorial design algorithm that assumes that the backbone stays fixed and results in a combination of mutations with a mostly additive effect on stability. In contrast, FuncLib/AbLift work in the regions of the protein system where positions are highly interdependent (the active/binding site). In such structural regions, there are fewer allowed mutations (⇐10% of the protein and very high conservation due to functional constraint) and almost all positions are dependent on one-another so there are almost no “safe” combinations of mutations, in which each mutation impacts activity in an additive way; they're all potentially deleterious, and indeed experiments show that these regions are incredibly sensitive to mutation, let alone multipoint mutations. Therefore, in the method provided herein, and implemented as the exemplary procedures FuncLib and AbLift, the tolerated sequence space is identified firstly, using more relaxed settings (energetic stability threshold) than PROSS, so as to enable mutations even in conserved positions, and secondly enumerates all of the possible combinations, which are kept at manageable numbers to enable effective computation. In each instance of a multipoint mutant generated by the method provided herein (FuncLib/AbLift), the backbone is allowed to change conformation, thereby allowing mutations, including small-to-large mutations that are considered very difficult for computational design and even combinations of small-to-large mutations. All of the enumerated multipoint mutants are then ranked by energy to ensure that only stable, pre-organised networks of mutations are selected. It has been surprisingly noticed by the inventors of the present invention, that there are often hundreds or even thousands of sequences with lower energies (more stable) than the wild type or the original/starting sequence, which has never been seen by applying straightforward combinatorial design simulations or in PROSS results. Thus, the method provided herein is based on a rigorous sampling of sequence space with fewer assumptions on the rigidity of the protein or on the additive contribution of mutations to function or stability.

While FuncLib and AbLift share many computational components, the main difference between the two implementation of the computational protein design method provided herein, is that FuncLib is mainly applied to enzyme active sites, which are solvent exposed and therefore potentially still tolerant to mutation, whereas AbLift is applied to the interface between two protein chains (e.g., light/heavy chain interface in antibodies). This chain interface region is as tightly packed as a protein core, and therefore potentially less tolerant to mutation. It is noted herein that PROSS, the previously provided method, typically fails to find mutations in such regions, and AbLift is designated to readily find hundreds of multipoint combinations with improved energy (stability and preorganization).

Hence, the method provided herein (FuncLib/AbLift) deals with the problem of how to find favourable multipoint mutants among interdependent positions in highly conserved regions—an outcome that PROS S explicitly tries to avoid, other computational design in general typically fail in, and experimental in vitro evolution strategies often require multiple iterative step-by-step screening in order to achieve.

Thus, according to an aspect of some embodiments of the present invention, there is provided a method for computationally designing a library of proteins (polypeptides), stemming from a template/original protein (original polypeptide chain), e.g., an enzyme, wherein members of this library exhibit 10-4,000 fold improvements in a range of activities and functionalities, compared to the template/original protein. In some embodiments, the protein is an enzyme with a known activity in terms of substrate/product/rate, and the library, which is generated according to embodiments of the present invention, include enzymes with either or both improved known activities, and/or new activities. It is noted that in the context of the present invention, a new activity may be seen as an activity known to be low or essentially null, hence the description below addresses both new and improved activities, as improvement can start from essentially no activity up to an enhanced activity, regardless of the known activity.

In terms of parameter values and Rosetta energy units, the more relaxed energetic stability threshold used in FuncLib/AbLift includes PSSM score ≥−2 or −1 and ΔΔG score ≤+1, +2, +3, +4, +5, or +6, compared to the energetic stability threshold used in PROSS, which includes PSSM score ≥0 and ΔΔG score ≤−0.45, −0.9, −2.0, −3.0, or −4.0.

For the demonstration of the method, the enzyme with a publically available crystal structure, zinc-containing phosphotriesterase (PTE) from Pseudomonas diminuta (PDB entry 1HZY), was selected. The method presented herein was effectively used to provide modified polypeptide chains, starting with an original polypeptide chain, such as found in a corresponding wild type protein or a previously engineered/designed variant, wherein several amino acid residues in the original polypeptide chains have been substituted such that a protein expressed to have the modified polypeptide chains (a variant protein) exhibits improved catalytic activity with respect to a certain substrate, as well as structural stability, compared to the wild type protein. The term “variant”, as used herein, refers to a designed protein obtained by employing the method presented herein. Herein and throughout, a terms “amino acid sequence” and/or “polypeptide chain” is used also as a reference to the protein having that amino acid sequence and/or that polypeptide chain; hence the terms “original amino acid sequence” and/or “original polypeptide chain” are equivalent or relate to the terms “original protein” and “wild type protein”, and the terms “modified amino acid sequence” and/or “modified polypeptide chain” and/or “designed polypeptide” are equivalent or relate to the terms “designed protein” and “variant”.

In some embodiments, the original polypeptide chain, or the original protein, is naturally occurring (wild type; WT) or artificial (man-made non-naturally occurring), or a designed polypeptide chain, namely a product of a computational method, such as PROSS.

In the context of some embodiments of the present invention, the term “designed” and any grammatical inflections thereof, refers to a non-naturally occurring sequence or protein.

In the context of some embodiments of the present invention, the term “sequence” is used interchangeably with the term “protein” when referring to a particular protein having the particular sequence.

According to an aspect of some embodiments of the present invention, there is provided a method of computationally designing a modified polypeptide chain starting from an original polypeptide chain.

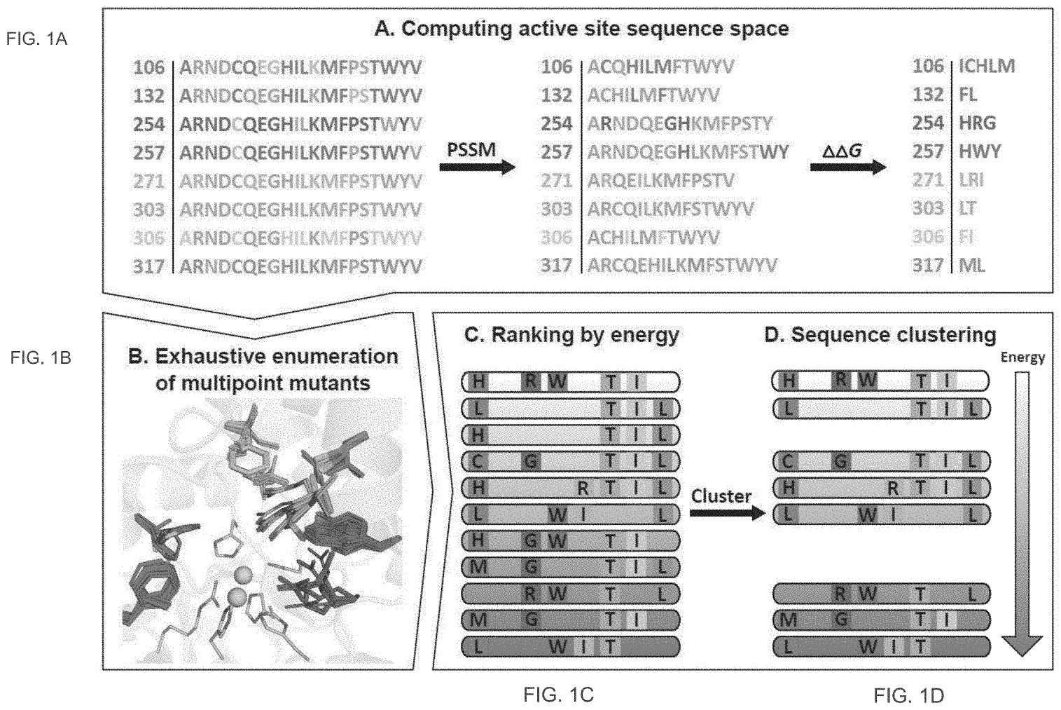

FIGS. 1A-D is a schematic illustration of an exemplary algorithm for executing the method of computationally designing a modified polypeptide chain starting from an original polypeptide chain, according to some embodiments of the present invention.

Method requirements and input preparation:

The basic requirements for implementing the method for designing modified polypeptide chains for activity diversification include:

availability of structural information pertaining to the original polypeptide chain, such as obtained from an experimentally determined crystal structure of the original polypeptide chain, or a crystal structure of a close homolog thereof, having at least 30-60% amino acid sequence identity, or computationally derived structural information based on an experimentally determined structure of a close homolog thereof;

optional availability of experimental mutation analysis, either point mutations, combinations of mutations, or deep mutational scanning; and

availability of sequence data derived from several qualifying homologous proteins, whereas the criteria for a qualifying homologous sequence are described below (FIG. 1A). In some cases of low availability of homologous proteins, the method utilizes a unique approach for selecting qualifying homologous sequences, as described below.

In the context of embodiments of the present invention, the term “% amino acid sequence identity” or in short “% identity” is used herein, as in the art, to describe the extent to which two amino acid sequences have the same residues at the same positions in an alignment. It is noted that the term “% identity” is also used in the context of nucleotide sequences.

It is noted herein that in general, the method presented herein (e.g., FuncLib) does not require a structural model of a transition state or its complex structure. Rather it computes diverse yet stable networks of interacting residues at the active-site pocket, thereby encoding different stereochemical complementarities for alternative substrates/ligands that do not need to be defined a priori. It is therefore expected that the method provides designs that form a functional repertoire, from which individual designs that efficiently turns-over various target substrates could be isolated. In applications that target a specific substrate, by contrast, sequence space can be further constrained by designing the enzyme in the presence of the substrate or transition-state model, and this option is enabled in the web-server, presented herein.

Structural data preparation:

According to some embodiments of the invention, the structural information is a set of atomic coordinates of the original polypeptide chain. This set of atomic coordinates is referred to herein as the “template structure”, which is used in the method as discussed below. In some embodiments, the template structure is a crystal structure of the original polypeptide chain, and in some embodiments the template structure is a computationally generated structure based on a crystal structure of a close homolog (more than 30-60% identity) of the original polypeptide chain, wherein the amino acid sequence of the original polypeptide chain has been threaded thereon and subjected to weighted fitting to afford energy minimization thereof, as these are discussed below.

In cases where the protein of interest is an oligomer (having several polypeptide chains), the chain of interest, or the original polypeptide chains to be modified, is defined in the template structure. In the case of hetero-oligomers, it is required to select the chain that will undergo the sequence design procedure or to subject both chains to simultaneous design. For homo-oligomers, it is advantageous to select the original polypeptide chain containing having more or better quality structural data. For example, in some homo-oligomers, binding ions may be discernible in a crystal structure in some of the chains and less so in others. In addition, it is advantageous to define key residues related to function and activity, as discussed hereinbelow.

Structure refinement:

According to some embodiments, prior to its use in the method presented herein, the template structure is optionally subjected to a global energy minimization, afforded by weighted fitting thereof, as discussed below.

According to some embodiments of the present invention, the template structure is optionally refined by energy minimization prior to using its coordinates, while fixing the conformations of key residues, as defined hereinbelow. Structure refinement is a routine procedure in computational chemistry, and typically involves weight fitting based on free energy minimization, subjected to rules, such as harmonic restraints.

The term “weight fitting”, according to some embodiments of any of the embodiment of the present invention, refers to a one or more computational structure refinement procedures or operations, aimed at optimizing geometrical, spatial and/or energy criteria by minimizing polynomial functions based on predetermined weights, restraints and constrains (constants) pertaining to, for example, sequence homology scores, backbone dihedral angles and/or atomic positions (variables) of the refined structure. According to some embodiments, a weight fitting procedure includes one or more of a modulation of bond lengths and angles, backbone dihedral (Ramachandran) angles, amino acid side-chain packing (rotamers) and an iterative substitution of an amino acid, whereas the terms “modulation of bond lengths and angles”, “modulation of backbone dihedral angles”, “amino acid side-chain packing” and “change of amino acid sequence” are also used herein to refer to, inter alia, well known optimization procedures and operations which are widely used in the field of computational chemistry and biology. An exemplary energy minimization procedure, according to some embodiments of the present invention, is the cyclic-coordinate descent (CCD), which can be implemented with the default all-atom energy function in the Rosetta™ software suite for macromolecular modeling. For a review of general optimization approaches, see for example, “Encyclopedia of Optimization” by Christodoulos A. Floudas and Panos M. Pardalos, Springer Pub., 2008.

According to some embodiments of the present invention, a suitable computational platform for executing the method presented herein is the Rosetta™ software suite platform, publically available from the “Rosetta@home” at the Baker laboratory, University of Washington, U.S.A. Briefly, Rosetta™ is a molecular modeling software package for understanding protein structures, protein design, protein docking, protein-DNA and protein-protein interactions. The Rosetta software contains multiple functional modules, including RosettaAbinitio, RosettaDesign, RosettaDock, RosettaAntibody, RosettaFragments, RosettaNMR, RosettaDNA, RosettaRNA, RosettaLigand, RosettaSymmetry, and more.

Weight fitting, according to some embodiments, is effected under a set of restraints, constrains and weights, referred to as rules. For example, when refining the backbone atomic positions and dihedral angles of any given polypeptide segment having a first conformation, so as to drive towards a different second conformation while attempting to preserve the dihedral angles observed in the second conformation as much as possible, the computational procedure would use harmonic restraints that bias, e.g., the Cα positions, and harmonic restraints that bias the backbone-dihedral angles from departing freely from those observed in the second conformation, hence allowing the minimal conformational change to take place per each structural determinant while driving the overall backbone to change into the second conformation.

In some embodiments, a global energy minimization is advantageous due to differences between the energy function that was used to determine and refine the source of the template structure, and the energy function used by the method presented herein. By allowing changes to occur in backbone conformation and in rotamer conformation through minimization, the global energy minimization relieves small mismatches and small steric clashes, thereby lowering the total free energy of some template structures by a significant amount.

In some embodiments, energy minimization may include iterations of rotamer sampling (repacking) followed by side chain and backbone minimization. An exemplary refinement protocol is provided in Korkegian, A. et al., Science, 2005. In some embodiments, energy minimization may include more substantial energy minimization in the backbone of the protein.

As used herein, the terms “rotamer sampling” and “repacking” refer to a particular weight fitting procedure wherein favorable side chain dihedral angles are sampled, as defined in the Rosetta software package. Repacking typically introduces larger structural changes to the weight fitted structure, compared to standard dihedral angles minimization, as the latter samples small changes in the residue conformation while repacking may swing a side chain around a dihedral angle such that it occupies an altogether different space in the protein structure.

In some embodiments, wherein the template structure is of a homologous protein, the query sequence is first threaded on the protein's template structure using well established computational procedures. For example, when using the Rosetta software package, according to some embodiments of the present invention, the first two iterations are done with a “soft” energy function wherein the atom radii are defined to be smaller. The use of smaller radius values reduces the strong repulsion forces resulting in a smoother energy landscape and allowing energy barriers to be crossed. The next iterations are done with the standard Rosetta energy function. A “coordinate constraint” term may be added to the standard energy function to allow substantial deviations from the original Cα coordinates. The coordinate constraint term behaves harmonically (Hooke's law), having a weight ranging between about 0.05-0.4 r.e.u (Rosetta energy units), depending on the degree of identity between the query sequence and the sequence of the template structure. During refinement, key residues are only subjected to small range minimization but not to rotamer sampling.

Sequence data preparation:

Once an original polypeptide chain has been identified, and a corresponding template structure has been provided, the method requires assembling a database of qualifying homologous amino acid sequences related to the amino acid sequence of the original polypeptide chain. The amino acid sequence of the original polypeptide chain can be extracted, for example, from a FASTA file that is typically available for proteins in the protein data bank (PDB), or provided otherwise. The search for qualifying homologous sequences is done, according to some embodiments of the present invention, in the non-redundant (nr) protein database, using the sequence of the original polypeptide chain as a search query. Such nr-database typically contains manually and automatically annotated sequences and is therefore much larger than databases that contain only manually annotated sequences.

A non-limiting examples of protein sequence databases include INSDC EMBL-Bank/DDBJ/GenBank nucleotide sequence databases, Ensembl, FlyBase (for the insect family Drosophilidae), H-Invitational Database (H-Inv), International Protein Index (IPI), Protein Information Resource (PIR-PSD), Protein Data Bank (PDB), Protein Research Foundation (PRF), RefSeq, Saccharomyces Genome Database (SGD), The Arabidopsis Information Resource (TAIR), TROME, UniProtKB/Swiss-Prot, UniProtKB/Swiss-Prot protein isoforms, UniProtKB/TrEMBL, Vertebrate and Genome Annotation Database (VEGA), WormBase, the European Patent Office (EPO), the Japan Patent Office (JPO) and the US Patent Office (USPTO).

A search in an nr-database yields variable results depending on the search query (amino-acid sequence of the original polypeptide chain). For proteins with lacking sequence data, results may include less than 10 hits. For proteins common to all life kingdoms the results may include thousands of hits. For most proteins, hundreds to thousands of hits are expected upon search in an nr-database. In all databases, including an nr-database and despite its name, there may be redundancy to some extent, and hits may be found in groups of identical sequences. The redundancy problem is addressed during the sequence data editing.

In some embodiments of the invention, the obtained sequence data is optionally filtered and edited as follows:

(a) Redundant sequences are clustered into a single representative sequence. The clustering is carried out with a predetermined threshold. For example, a threshold of 0.97 means that all sequences that share at least 97% identity among themselves are clustered into a single representative sequence that is the average of all the sequences contributing to the cluster;

(b) Sequences for which the alignment length is less than a predetermined threshold (e.g., 60%) of the search query length are excluded; and

(c) Sequences that exhibit less than about 28% to 34% identity cutoff, for example, with respect to the search query are excluded, following guidelines such as provided elsewhere [Rost, B., Protein Eng, 1999, 12(2):85-94].

The exact choice of the minimal identity parameter depends on the richness of the sequence data. Hence, according to some embodiments of the invention, if the number of sequence hits afforded under a strict threshold is about 50 or less, a less strict threshold may be used (lower % identity). The effect of threshold tuning of the identity parameter is demonstrated in the design of a phosphotriesterase from pseudomonas diminuta, where lowering the threshold from 30% to 28% identity increased the number of qualifying homologous sequences from 45 to 95.

In some embodiments of the invention, the cutoff for electing qualifying homologous sequences for a multiple sequence alignment is more than 20%, 25%, 30%, 35%, 40%, or more than 50% identity with respect to the original polypeptide chain.

It is noted that the method is not limited to any particular sequence database, search method, identity determination algorithm, and any set of criteria for qualifying homologous sequences. However, the quality of the results obtained by use of the method depends to some extent on the quality of the input sequence data.

Once an assembly of qualifying homologous sequences is obtained, a multiple sequence alignment (MSA) is generated (FIG. 1A), typically by using a designated multiple sequence alignment algorithm, such as that implemented in MUSCLE [Edgar, R. C., Nucleic Acids Res, 2004, 32(5): 1792-1797]. Alternatively, a Basic Local Alignment Search Tool (BLAST) can be used to generate MSA files.

Cases of low availability of homologous proteins:

Generally, adding sequences exhibiting a % identity below 20% to a MSA having dozens of homologous sequences of higher % identity may contribute diversity to the alignment; however, adding such kind of low % identity sequences increases the risk of errors (false positives) significantly while not necessarily improving diversity by much, since most of this diversity will probably be covered by the high homology sequences that were already part of the MSA. On the other hand, when the protein of interest is poorly represented in the sequence database, using a low % identity homolog becomes an advantage rather than a risk.

In some cases the protein of interest is poorly represented in the currently available protein sequence databases in terms of the number of non-redundant homologous sequences. For example, in case that a sequence homology search finds only one homologous sequence having 60% sequence identity to the protein of interest, that means that the method is limited to zero amino acid substitutions in 60% of the sequence positions, and out of the remaining 40% it would have been difficult to identify a position with more than few amino acid alternatives.

In such cases, the present inventors have envisioned several scenarios where standard sequence homology search methods might result in low sequence diversity within the space of homologous sequences (e.g., less than 50%, less than 40%, less than 30%, less than 25% (the “twilight zone”) or less than 20% sequence identity with respect to the amino acid sequence of the protein of interest). An example for such a scenario is where the fold of the protein of interest (the target protein, also referred to herein as the original polypeptide chain) is unique or phylogenetically restricted to particular genera or phyla, or the protein function has emerged in recent millennia and the protein of interest therefore has few homologues. It was envisioned by the present inventors that in such or other cases of low sequence diversity, the following steps could be taken to increase the sequence diversity used by presently provided method, while minimizing the risk of introducing unrelated sequences.

An exemplary sub-algorithm for treating such cases is described in U.S. Patent Application Publication No. 2017/0032079, which is incorporated herein by reference. The general rational behind this sub-algorithm is to increase the number of homologous sequences in the MSA as much as possible while minimizing the risk of including non-related sequences; for example, accounting for the fact that the fold of the protein of interest is unique and/or phylogenetically distant from typical organisms interrogated by sequencing efforts.

Step 1: search for low-sequence identity homologous sequences (e.g., less than 50%, less than 40%, less than 30%, less than 25% or less than 20% sequence identity; preferably less than 30% identity) in any given sequence database by using an algorithm that specializes in detection of distant homologues (e.g., CSI-BLAST; see, PMIDs: 19234132, 18004781);

Step 2: cluster the results from Step 1 using a clustering threshold 90-100% (see, e.g., PMID: 11294794);

Step 3: remove sequences with coverage below 40% relative to that of the original polypeptide chain (protein of interest), and sequence identity of less than 15%;

Step 4: inspect the annotation and source organism of each sequence in the list resulting from Step 3, and exclude sequences that have a high chance of being false positives. Non-limiting examples are hits that have no molecular-function annotation (typically these are annotated as “hypothetical protein”), sequences from genera or phyla other than the protein of interest's genus or phylum, or proteins that are annotated with functions that are different from the function of the protein of interest;

Step 5 Exclude sequences that have more than 5%, more than 4%, more than 3%, more than 2%, more than 1%, or more than 0.5% gaps (insertions or deletions, known by the acronym INDELs), preferably less than 5% gaps in a pairwise alignment with the original polypeptide chain (see, e.g., PMID: 18048315); Step 6: Combine sequences resulting from Step 5 with high sequence identity sequences (i.e., more than 30% sequence identity to the protein of interest) that were collected and processed using any sequence identity search protocol, and generate a multiple-sequence alignment (MSA). This MSA can then be used as input by the method presented herein even if it contains few (less than 3-10) sequences.

Following is a More Specific Yet Non-Limiting Example:

Step I: Use the CSI-BLAST search algorithm instead of BLASTP to identify homologs. The use of an alternative sequence search algorithm to find distant homologues, such as using CSI-BLAST (context-specific iterative BLAST) with 3 iterations instead of BLASTP is advantageous in some cases since CSI-BLAST constructs a different substitution matrix to calculate alignment scores. The CSI-BLAST matrix is context specific (i.e., each position probabilities depend also on 12 neighboring amino acids), thus it finds 50% more homologous sequences than BLAST at the same error rate. The iterative use means that this process is repeated and at the end of each round the substitution matrix is updated according the sequence information from homologues collected up to that point.

Step II: Use minimal sequence identity thresholds of 19% and 15% for strict and permissive alignments respectively. Lowering the minimal sequence identity threshold to 15% (permissive alignment) and 19%, (strict alignment) while using BLASTP may be meaningless since BLASTP is tuned to find sequences with higher sequence identity to the target. Secondly, these thresholds are chosen according to the results obtained from the CSI-BLAST search; hence these thresholds are set after the CSI-BLAST search and depend on outcome; specifically, the thresholds may need to be adjusted to obtain more true positive or fewer false positive hits, where true positive are hits with a functional annotation and phylogenetic origin that correspond to the requirements of Step III, below.

Step III: Exclude sequences from genera or phyla other than the one corresponding to the protein of interest if it is expected that protein target's fold or function are unique to the genus of phylum of the target protein. If this expectation holds, proteins from genera and phyla outside those of the target protein are likely to be false-positive hits; that is, proteins that adopt different folds or function.

Step IV: Use an INDEL fraction of up to 1% for sequences sharing below 19% sequence identity, in pairwise alignment with the query. In the treatment of gaps/INDELs, the CSI-BLAST pairwise alignment INDELS fraction may be required to be up to 1% for sequence with minimal % identity below 19%. The rationale is that for low-homology sequences sharing such a small sequence identity to the query, the risk of inserting false positives in the MSA is too high, but a small INDEL fraction indicates that these are likely to be true hits.

Step V: Use sequence coverage threshold for hits relative to the target protein in the alignment to 50%. It is likely that all the sequences that passed the criteria set forth in Steps II, III and IV will exhibit a coverage of more than 50%; however, if the coverage threshold is set to 60%, as typically practiced in the art, most of the sequences would be filtered out.

Step VI: Generate MSA for the remaining sequences as typically practiced in the art.

Variable loop regions:

BLAST algorithms may provide results that include sequences with different lengths. The differences typically stem from different lengths in loop regions, and loops with different lengths may reflect different biochemical context. As a result, MSA columns representing loop positions may contain aligned residues from loops with different length, thus possibly degrading the data with information from different biochemical context, possibly irrelevant to the biochemical context of the protein of interest. A BLAST hit may therefore contain relevant information at some positions while containing non-relevant information in other positions. To minimize the level of irrelevant sequence information for each loop, the secondary structure of the original protein is identified and a context specific sub-MSA file is created for each loop region, and the sub-MSA contains only loop sequences with the same length.

Secondary structure identification is done through identification of hydrogen bond patterns in the structure and this is termed “dictionary of protein secondary structure” (DSSP). There are several software packages available that offer such analysis, such as, for example, a Rosetta™ module for loop identification.

The output of the secondary structure identification procedure is typically a string (i.e., an output string) that has the same length as the template structure, wherein each character represents a residue in a secondary structure element that may be either H, E or L, denoting an amino acid forming a part of either an a-helix, a β-sheet or a loop.

According to some embodiments of the invention, the amino acid sequence of the loop regions in the structure of the original protein is processed as follows:

(a) Loops in the template structure are identified by automatic or manual inspection of a structure model, and/or by any secondary-structure analyzing algorithms.

(b) The positions representing each loop on the output string are determined including loop stems (two additional amino acids at each end of the loop). To account for the stems, two positions are added to each of the loop's ends, unless the loop is at one of the main-chain termini. According to some embodiments of the invention, it is advantageous to include the stems in the loop definition since stems anchoring different loops may potentially exhibit different conformations and form different contacts among themselves or with the loop residues, and it is advantageous that the sequence data used as input in the method presented would represent that.

For example, if the secondary structure output string is:

LLLHHHHHHHLLLLLHHHHHLLLEEEE

then the loop regions are defined at positions 1-5, 9-17 and 19-25 (bold characters).

(c) The positions that represent each loop are identified in the query sequence in the MSA. The loop positions in the MSA may be different than the loop positions in the original string from the previous step since in the MSA the query is aligned to other sequences and may therefore contain both amino acid characters and hyphens, representing gaps.

(d) After the loop positions were located on the query sequence in the MSA, a character pattern is defined for each loop. For example, a pattern may comprise “X” character to represent an amino acid and “-” (hyphen) to represent a gap.

(e) Lastly, a context specific sub-MSA file is generated for each loop excluding all sequences that do not share the same character pattern for that loop, namely context specific sub-MSA contains sequences wherein the loop has the same length, gaps included.

For example, positions 4-10 in a hypothetical original protein are recognized as a loop with the hypothetical sequence “APTESVV” including stems. The loop is identified on the query protein in the MSA file and the pattern is found to be “A—PTESVV”. The context specific sub-MSA file that will be generated for this loop with all the sequences in the MSA file will contain the pattern “X—XXXXX”.

Thus, according to some embodiments of the present invention, for loop regions, the sequence alignment comprises amino acid sequences having sequence length equal to a corresponding loop in the original polypeptide chain. Accordingly, sequence alignments, which are relevant in the context of loop regions, are referred to herein as “context specific sub-MSA”.

Rules for substitutions:

The method calls for identification of substitutable residues. The selection of substitutable residues may rely on expert-guided decision on positions to mutate. These positions are typically positions in the active site of an enzyme that are not crucial for the core catalytic activity but are in proximity (first shell) of the substrate or in proximity to first shell positions (second shell) etc.

In some embodiments of the present invention, a set of restraints, constrains and weights are used as rules that govern some of the computational procedures. In the context of some embodiments of the present invention, these rules are applied in the method presented herein to determine which of the positions in the original polypeptide chain will be allowed to permute (be substituted), and to which amino acid alternative. These rules may also be used to preserve, at least to some extent, some positions in the sequence of the original polypeptide chain.

One of the rules employed in amino acid sequence alterations stem from highly conserved sequence patterns at specific positions, which are typically exhibited in families of structurally similar proteins. According to some embodiments of the present invention, the rules by which a substitution of amino acids is dictated during a sequence design procedure include position-specific scoring matrix values, or PSSMs.

A “position-specific scoring matrix” (PSSM), also known in the art as position weight matrix (PWM), or a position-specific weight matrix (PSWM), is a commonly used representation of recurring patterns in biological sequences, based on the frequency of appearance of a character (monomer; amino acid; nucleic acid etc.) in a given position along the sequence. Thus, PSSM represents the log-likelihood of observing mutations to any of the 20 amino acids at each position. PSSMs are often derived from a set of aligned sequences that are thought to be structurally and functionally related and have become widely used in many software tools for computational motif discovery. In the context of amino acid sequences, a PSSM is a type of scoring matrix used in protein BLAST searches in which amino acid substitution scores are given separately for each position in a protein multiple sequence alignment. Thus, a Tyr-Trp substitution at position A of an alignment may receive a very different score than the same substitution at position B, subject to different levels of amino acid conservation at the two positions. This is in contrast to position-independent matrices such as the PAM and BLOSUM matrices, in which the Tyr-Trp substitution receives the same score no matter at what position it occurs. PSSM scores are generally shown as positive or negative integers. Positive scores indicate that the given amino acid substitution occurs more frequently in the alignment than expected by chance, while negative scores indicate that the substitution occurs less frequently than expected. Large positive scores often indicate critical functional residues, which may be active site residues or residues required for other intermolecular or intramolecular interactions. PSSMs can be created using Position-Specific Iterative Basic Local Alignment Search Tool (PSI-BLAST) [Schäffer, A. A. et al., Nucl. Acids Res., 2001, 29(14), pp. 2994-3005], which finds similar protein sequences to a query sequence, and then constructs a PSSM from the resulting alignment. Alternatively, PSSMs can be retrieved from the National Center for Biotechnology Information Conserved Domains Database (NCBI CDD) database, since each conserved domain is represented by a PSSM that encodes the observed substitutions in the seed alignments. These CD records can be found either by text searching in Entrez Conserved Domains or by using Reverse Position-Specific BLAST (RPS-BLAST), also known as CD-Search, to locate these domains on an input protein sequence.

In the context of some embodiments of the present invention, a PSSM data file can be in the form of a table of integers, each indicating how evolutionary conserved is any one of the 20 amino acids at any possible position in the sequence of the designed protein. As indicated hereinabove, a positive integer indicates that an amino acid is more probable in the given position than it would have been in a random position in a random protein, and a negative integer indicates that an amino acid is less probable at the given position than it would have been in a random protein. In general, the PSSM scores are determined according to a combination of the information in the input MSA and general information about amino acid substitutions in nature, as introduced, for example, by the BLOSUM62 matrix [Eddy, S. R., Nat Biotechnol, 2004, 22(8), pp. 1035-6].

In general, the method presented herein can use the PSSM output of a PSI-BLAST software package to derive a PSSM for both the original MSA and all sub-MSA files. A final PSSM input file, according to some embodiments of the present invention, includes the relevant lines from each PSSM file. For sequence positions that represent a secondary structure, relevant lines are copied from the PSSM derived from the original full MSA. For each loop, relevant lines are copied from the PSSM derived from the sub-MSA file representing that loop. Thus, according to some embodiments of the present invention, a final PSSM input file is a quantitative representation of the sequence data, which is incorporated in the structural calculations, as discussed hereinbelow.

According to some embodiments of the present invention, MSA and PSSM-based rules determine the unsubstitutable positions and the substitutable positions in the amino acid sequence of the original polypeptide chain, and further determine which of the amino acid alternatives will serve as candidate alternatives in the single position scanning step of the method, as discussed hereinbelow.

Key residues:

The method, according to some embodiments of the present invention, allows the incorporation of information about the original polypeptide chain and/or the wild type protein. This information, which can be provided by various sources, in incorporated into the method as part of the rules by which amino acid substitutions are governed during the design procedure. Albeit optional, the addition of such information is advantageous as it reduces the probability of the method providing results which include folding- and/or function-abrogating substitutions. In the examples presented in the Example section below, valuable information about activity has been employed successfully as part of the rules.

The term “key residues” refer to positions in the designed sequence that are defined in the rules as fixed (invariable), at least to some extent. Sequence positions, which are occupied by key residues optionally, constitute a part of the unsubstitutable positions.

Information pertaining to key residues can be extracted, for example, from the structure of the original polypeptide chain (or the template structure), or from other highly similar structures when available. Exemplary criteria that can assist in identifying key residues, and support reasoning for fixing an amino-acid type or identity at any given position, include:

In the previous provided protein stabilization design method, PROSS, when used to provide stabilized enzyme variants, the key residues are selected within a radius of about 5-8 Å around the substrate binding site, as may be inferred from complex crystal structures comprising a substrate, a substrate analog, an inhibitor and the like. Similarly, when using PROSS to provide stabilized metal binding proteins, key residues are selected within about 5-8 Å around a metal atom. Other key residues may be designated in protein interface that involves the chain of interest in an oligomers, as interacting chains are oftentimes involved in dimerization interfaces, binding ligands or protein-substrates interactions. Likewise, key residues may be designated within a certain distance from DNA/RNA chains interacting with the protein of interest, within a certain distance from an epitope region, and the likes.

It is noted that the shape and size of the space within which key residues are selected is not limited to a sphere of a radius of 5-8 Å; the space can be of any size and shape that corresponds to the sequence, function and structure of the original protein. It is further noted that specific key residues may be provided by any external source of information (e.g., a researcher).

In the context of the present invention, key residues are selected sparingly (≤10 positions, and more typically 0-3 positions), even and particularly in and around regions of the activity the method is attempting to diversify or improve. This strategy allows the activity-determining regions to diversify while the stability of the protein is not sacrificed.

When the template structure, the PSSM file (which is based on the full MSA and any optional context specific sub-MSA), and the identification of key residues, unsubstitutable positions and the substitutable positions are provided, the method presented herein can use these data to provide the modified polypeptide chain starting from the original polypeptide chain.

Main method steps:

The objective of the method provided herein (FuncLib/AbLIFT) is to design a small set of stable, efficient, and functionally diverse multipoint active-site mutants suitable for low-throughput experimental testing. The design strategy is general and can be applied, in principle, to any natural enzyme or designed protein, using its molecular structure and a diverse set of homologous sequences.

According to some embodiments of the present invention, the method presented herein includes a step that determines which of the positions in the amino-acid sequence of the original polypeptide chain will be subjected to amino-acid substitution and which amino acid alternatives will be assessed. (referred to herein as substitutable positions), and in which positions in the amino acid sequence of the original polypeptide chain the amino-acid will not be subjected to amino-acid substitution (referred to herein as unsubstitutable positions).

In a following step, (single position scanning step), a position-specific stability score is given to each of the allowed amino acid alternatives at each substitutable position. In the enzyme repertoire cases, the active-site residues were defined to be designed by visual examination of the enzyme molecular structures. Evolutionary conservation scores were computed from PSSMs and ΔΔG values were computed essentially as described previously [Goldenzweig, A. et al. Mol Cell., 2016, 63(2), pp. 337-346]. Tolerated amino acid identities at the active site of PTE were filtered according to the following thresholds: PSSM≥−2 and ΔΔG≤+6 R.e.u.

It is noted that the detailed description of the method presented herein is using some terms, units and procedures with are common or unique to the Rosetta™ software package, however, it is to be understood that the method is capable of being implemented using other software modules and packages, and other terms, units and procedures are therefore contemplated within the scope of the present invention.

It is also noted that the detailed description of the method presented herein is using the proteins and variables presented in the Examples section, which are not to be seen as limiting in any way, as the method is applicable for any protein and polypeptide chain sequence for which the required data is available.

According to some embodiments of the present invention, the following step of the method is an exhaustive enumeration of all possible combinations of at least 3 and as many as 5, 6, 7, 8, 9, 10 or more six mutations in the original polypeptide chain (e.g. of PTE). Each mutant was modeled in Rosetta, including combinatorial sidechain packing, and the backbone and sidechains of all residues were minimized energetically, subject to harmonic restraints on the Cα coordinates of the entire protein (being composed of one polypeptide chain or more). All designed polypeptide chains (designed proteins, or “designs” for short) were ranked according to all-atom energy, and the top-ranked designs were chosen for experimental analysis after removing designs with fewer than two mutations relative to one another.

As stated hereinabove, one of the main differences between PROSS and the method provided herein is the combinatorial design step in PROSS that is being replaced by a comprehensive enumeration step in the instant method. In the exemplary study presented here, small-scale testing of the method provided herein (FuncLib/AbLift) proved sufficient to identify variants that exhibited orders-of-magnitude changes in enzyme activity profiles without loss in apparent protein stability. The method can therefore be used to rapidly optimize specific activities or generate functional repertoires from enzymes that are not amenable to high-throughput screening. Whereas conventional active-site design strategies rely on transition-state modeling, the method provided herein computes diverse and stable networks of interacting active-site mutations, enabling design even in the cases discussed here, for which enzyme transition-state models are uncertain. Although the designed mutations conserve the wild type backbone structure, some designs exhibit sign-epistatic relationships, which render these designs all but inaccessible to stepwise mutational trajectories. Thus, the sequence space of an enzyme active site provides a vast resource of functional diversity that defies exploration by natural and laboratory evolution but can now be accessed through computational protein design.

According to some embodiments of the present invention, the method is implemented effectively for original polypeptide chains that comprise more than 100 amino acids (aa). In some embodiments, the original polypeptide chains comprise more than 110 aa, more than 120 aa, more than 130 aa, more than 140 aa, more than 150 aa, more than 160 aa, more than 170 aa, more than 180 aa, more than 190 aa, more than 200 aa, more than 210 aa, more than 220 aa, more than 230 aa, more than 240 aa, more than 250 aa, more than 260 aa, more than 270 aa, more than 280 aa, more than 290 aa, more than 300 aa, more than 350 aa, more than 400 aa, more than 450 aa, more than 500 aa, more than 550 aa, or more than 600 amino acids.

According to some embodiments of the present invention, the method presented herein provides modified polypeptide chains having more than 2 amino acid substitutions (mutations), more than 3 substitutions, more than 4 substitutions, more than 5 amino acid substitutions, more than 6 substitutions, more than 7 substitutions, more than 8 substitutions, more than 9 substitutions, more than 10 substitutions, more than 11 substitutions, or more than 12 substitutions compared to the starting original polypeptide chain.

Sequence space:

According to some embodiments of the present invention, after filtering key residues and imposing a free energy acceptance threshold, the number of substitutable positions in a given sequence is greatly reduced, thereby providing a wide yet manageable combinatorial sequence space from which designed sequences can be selected. Thus, the term “sequence space” refers to a set of substitutable positions, each having at least one optional substitution over the original/WT amino acid at the given position.

A sequence space is therefore a result of a certain acceptance threshold; each acceptance threshold produces a different sequence space, where sequence spaces defined by stricter acceptance thresholds are contained within larger sequence spaces defined by more permissive acceptance thresholds. As discussed hereinabove, in order to avoid false positives the acceptance threshold can be small and should be negative, wherein −2 r.e.u is considered to be highly restrictive (strict) and +6 r.e.u is highly permissive. The sequence space obtained by using acceptance threshold of +6 r.e.u will inevitably be larger (permissive) than a sequence space obtained by using acceptance threshold of −2.00 r.e.u (strict). Experimental use of the method presented herein to produce actual proteins has shown that an intermediate acceptance threshold produces an optimal sequence space. In fact, the sequence space is a sub-space of the broader space defined by the PSSM rules.

An exemplary and general means to present a sequence space is in a list of sequence positions based on the wild-type sequence numbering, P₁, P₂, P3, . . . , Pn, wherein each position is either designated as a key residue, namely an amino acid as found in the WT, AA_{WT}; or a position that can take any one amino acid from a limited list comprising at least one alternative amino acid based on the PSSM and energy minimization analysis, AAₘ, wherein m is a number denoting one of the naturally occurring amino acids, e.g., A=1, R=2, N=3, D=4, C=5, Q=6, E=7, G=8, H=9, L=10, I=11, K=12, M=13, F=14, P=15, S=16, T=17, W=18, Y=19 and V=20 (aa numbering is arbitrary and used herein to demonstrate a general representation of a sequence space.

For example, the sequence space can be presented as:

P₁: AA_{WT}, AA₅, AA₈, and AA₁₂;

P₂: AA_{WT};

P₃: AA_{WT} and AA₁₆;

P₄: AA_{WT}, AA₁, AA₃, AA₆, AA₁₀, and AA₁₄;

P₅: AA_{WT}, AA₄, AA₈, and AA₁₁;

. . .

Pn: AA_{WT}, AAₘ, AAₘ, AAₘ, AAₘ, and AAₘ,;

whereas in this general example, P₁ has four alternative amino acids, P₂ is a key residue and so forth.

According to some embodiments of the present invention, the sequence space can be further limited by imposing a stricter acceptance threshold, or expanded by imposing a more permissive acceptance threshold. In general, the value of +2 r.e.u has been found to be adequately permissive; however sequence space based on an acceptance threshold larger than +2 r.e.u (e.g., +6 r.e.u) or based on an acceptance threshold smaller than −2.00 r.e.u (e.g., −2.1 r.e.u) are also contemplated.

In the Examples section that follows below, a sequence space based on acceptance threshold of +6 r.e.u is presented for some of the exemplary proteins on which the method has been demonstrated. Any designed sequence having any choice of any 2 or more substitutions relative to the wild-type/starting sequence that are selected from the presented sequence space, and that exhibits, at least one improved catalytic activity, is contemplated within the scope of the present invention.

It is noted herein that embodiments of the present invention encompass any and all the possible combinations of amino acid alternatives in any given sequence space afforded by the method presented herein (all possible variants stemming from the sequence space as defined herein).

It is further noted that in some embodiments of the present invention, the sequence space resulting from implementation of the method presented herein on an original protein, can be applied on another protein that is different than the original protein, as long as the other protein exhibits at least 30%, at least 40%, or at least 50% sequence identity and higher. For example, a set of amino acid alternatives, taken from a sequence space afforded by implementing the method presented herein on a human protein, can be used to modify a non-human protein by producing a variant of the non-human protein having amino acid substitutions at the sequence-equivalent positions. The resulting variant of the non-human protein, referred to herein as a “hybrid variant”, would then have “human amino acid substitutions” (selected from a sequence space afforded for a human protein) at positions that align with the corresponding position in the human protein. In some embodiments of the present invention, any such hybrid variant, having at least 2 substitutions that match amino acid alternatives in any given sequence space afforded by the method presented herein (all possible variants stemming from the sequence space as defined herein), is contemplated and encompassed in the scope of the present invention.

FuncLib web-server:

A FuncLib web-server was constructed to implement several improvements of the method presented herein. In designing the exemplary enzyme PTE variants, as presented herein, a multiple-sequence alignment (MSA) was computed for the entire protein sequence, and wherever loops were observed in the query structure, any aligned sequence that exhibited gaps relative to the query was eliminated to reduce alignment ambiguity (see [Goldenzweig, A. et al.. Mol Cell., 2016, 63(2), pp. 337-346]). In the FuncLib web-server, by contrast, all secondary-structure elements are subjected to this filtering, resulting in improved PSSM accuracy, particularly in the active-site pocket. Furthermore, the web-server implements more accurate atomistic modeling and scoring: it uses the recent Rosetta energy function [Park, H. et al., J Chem Theory Comput., 2016, 12(12), pp. 6201-6212] with improved electrostatics and solvation potentials relative to previous Rosetta energy functions; implements harmonic coordinate restraints on sidechain atoms of essential amino acid residues in the catalytic pocket to guarantee their preorganization; restricts refinement to amino acids within 8 Å (or within the range of 6-10 Å) of designed positions instead of refining the entire protein; allows the user to modify the tolerated sequence space (for instance, based on prior experimental and structural analysis); and enables modeling of small-molecule ligands or transition-state complexes.

Diverse phosphotriesterase repertoire:

Natural and laboratory evolution of altered activities depend on the stepwise accumulation of mutations, each of which must be at least neutral in fitness. Following a few mutations, however, improvements in activity often plateau due to epistasis or stability-threshold effects. Typical evolutionary trajectories leading from one highly efficient enzyme to another are therefore time-consuming and often comprise dozens of enabling mutations outside the active site, most of which only contribute to the activity indirectly, for instance by stabilizing the enzyme. The strategy presented herein rationalizes and accelerates the generation of stable enzymes exhibiting altered activities: it starts by designing stable and highly expressed enzyme variants, using a method provided previously (PROSS), and then designs dozens of variants that encode preorganized networks of active-site mutants exhibiting different stereochemical features. The combination of evolutionary-conservation analysis and Rosetta atomistic modeling focuses design calculations on stable, preorganized, and functional active-site constellations.

Accordingly, the present inventors have implemented the FuncLib procedure in order to enumerate PTE variants with enhanced catalytic activities towards substrates, towards which WT PTE is less effective, as such PTE variants could serve as a detoxification agent against various organophosphate/nerve agents, as well as to increase PTE's catalytic activity towards known PTE substrates, such as VX type nerve agent. Using a PROSS-stabilized sequence [WO 2017/017673; Goldenzweig, A. et al.. Mol Cell., 2016, 63(2), pp. 337-346] dPTE2 (SEQ ID NO: 1), which is a variant of PTE that contained 20 mutations outside the active-site pocket and stemming from PTE-S5 [Roodveldt, C. and Tawfik, D.S., Protein Eng Des Sel., 2005, 18(1), pp. 51-8], and using the crystal structure of WT PTE (PDB Entry: 1HZY), the designed variants obtained by the method presented herein exhibited broad spectrum activity having thousands-folds activity relative to WT PTE.

Thus, according to one aspect of the invention there is provided a protein having a sequence selected from the group consisting of any combination of at least 2 amino acid substitutions of a sequence space afforded for phosphotriesterase (PTE) from Pseudomonas diminuta as an original protein, and listed in Table A blow, whereas wild type positons, I106, F132, H254, H257, L271, L303, F306 and M317, are not shown therein.

TABLE A
Position (numbering according to PDB entry: 1HZY

| 106 | 132 | 254 | 257 | 271 | 303 | 306 | 317 |
|---|---|---|---|---|---|---|---|
| C/H/L/M | L | G/R | Y/W | I/R | T | I | L |

The protein, according to some embodiments of the present invention, can be selected from the list presented in Table A set forth herein. In some embodiments the protein has a sequence selected from the group consisting of PTE_28 (SEQ ID NO: 28), PTE_29 (SEQ ID NO: 29), PTE_56 (SEQ ID NO: 56), and PTE_57 (SEQ ID NO: 57).

According to some embodiments, the protein can be an isolated protein, a fusion to another domain, such as Fc, or a mixture of proteins and other agents, factors carriers and the likes, as long as it includes at least one of the PTE designed proteins, as defined in Table A.

The original protein can be any enzyme of the PTE family having the EC No. 3.1.8.1 (EC: 3.1.8.1), including wild-type PTE from Pseudomonas diminuta or any other biological, or any designed of artificial PTE, including PTE variants obtained by using a computational method, such as, but not limited to, PROSS. In order to identify the amino acid residues for substitution of any original protein, the sequence of the original protein is aligned with the sequence of phosphotriesterase (PTE) from Pseudomonas diminuta as presented in PDB entry: 1HZY. As used herein, the term “phosphotriesterase” abbreviated herein to PTE, also referred to as Parathion hydrolase (EC: 3.1.8.1), refers to an enzyme belonging to the amidohydrolase superfamily. The phosphotriesterases of this aspect of the present invention are bacterial phosphotriesterases that have an enhanced catalytic activity towards V-type organophosphonates due to an extended loop 7 amino acid sequence, as compared to other phosphotriesterases. Such phosphotriesterases have been identified in Brevundimonas diminuta, Flavobacterium sp. (PTEflavob) and Agrobacterium sp.

As used herein, a “nerve agent” refers to an organophosphate (OP) compound such as having an acetylcholinesterase inhibitory activity. The toxicity of an OP compound depends on the rate of its inhibition of acetylcholinesterase with the concomitant release of the leaving group such as fluoride, alkylthiolate, cyanide or aryoxy group. The nerve agent may be a racemic composition or a purified enantiomer (e.g., Sp or Rp). In the context of embodiments of the present invention, the terms “organophosphate” or “nerve agent” encompass V-type (Amiton) nerve agent, G-type (Trilon) nerve agents and GV-type (Novichok) nerve agents. In the context of embodiments of the present invention, the term “nerve agent” includes, without limitation, G-type agents such as Tabun (GA), Sarin (GB), Chlorosarin (GC), Soman (GD), Ethylsarin (GE), and Cyclosarin (GF), V-type agents such as EA-3148, VE, VG, VM, VP, VR, VS, R/S-VX, CVX and RVX, and GV-type such as Novichok agents and GV (2- [dimethylamino(fluoro)phosphoryl]-N,N-dimethylethanamine).

A method of organophosphate detoxification:

According to an aspect of the present invention, the designed proteins, or PTE variants provided herein, can be used for decontamination of equipment, clothes and environment by hydrolyzing a broad spectrum of organophosphate agents, including nerve agents from the G-type, V-type, and GV-type nerve agents, and thereby detoxify an object or an area which is suspected of being contaminated with such agents. The area can be an inanimate object, a ground, a piece of equipment, a piece of clothing and a bodily surface.

In some embodiments, the designed proteins, or PTE variants provided herein, can be administered in vivo to a subject being suspected of nerve agent poisoning. In such uses, the protein is administered as a pharmaceutical composition, and may include a pharmaceutically accepted carrier as well as other active ingredients and excipients.

It is expected that during the life of a patent maturing from this application many relevant designed PTE variants with broad specificity hydrolysis of organophosphates will be developed and the scope of the phrase “designed PTE variants” is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: # is expressed in a DNA sequence format (e.g., reciting T for thymine), but it can refer to either a DNA sequence that corresponds to an # nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental or calculated support in the following examples.

### EXAMPLES

Reference is now made to the following examples, which together with the above descriptions illustrate some embodiments of the invention in a non-limiting fashion.

### Example 1

### Computational Method

Embodiments of the present platform, also termed as FuncLib, aim at the design of a small set of stable, efficient, and functionally diverse multipoint active-site mutants suitable for low-throughput experimental testing. The design strategy is general and can be applied, in principle, to any natural enzyme using its molecular structure and a diverse set of homologous sequences (FIGS. 1A-D).

Computational tools:

The Rosetta software suite for biomolecular design was used as the framework for the computational part of the method, and is available for download at www(dot)rosettacommons(dot)org. Specifically, the Rosetta GitHub version 627f7dd22223c3074594934b789abb4f4e2e3b10 was used for all simulations. All Rosetta modeling and design was done using RosettaScripts [Fleishman, S. L. et al., PLoS One, 2011, 6(6)], which are available with their command lines and flag files herein below. All design calculations used the Rosetta talaris14 all-atom energy function, which is dominated by van der Waals packing, hydrogen bonding, solvation, and electrostatics.

FuncLib design strategy:

The objective of the method provided herein (FuncLib) was to design a small set of stable, efficient, and functionally diverse multipoint active-site variants (mutants) suitable for low-throughput experimental testing. The design strategy, which was used, is general and can be applied to any natural enzyme or designed protein, using its molecular structure and a diverse set of homologous sequences.

FIGS. 1A-C presents a schematic flow chart illustrating key steps in the method for producing a library of functional designs of a given enzyme. For example only and without limitation, FIGS. 1A-C illustrate steps in the generation of a repertoire of phosphotriesterase (PTE) enzymes starting from the crystal structure of a bacterial phosphotriesterase (PTE; PDB entry: 1HZY) and the sequence of a PROSS-stabilized variant of PTE, dPTE2 (SEQ ID NO: 1). Specifically, FIG. 1A shows the step wherein active-site positions are selected for design, and at each position, sequence space is constrained by evolutionary-conservation analysis (PSSM) and mutational-scanning calculations (ΔΔG). FIG. 1B shows the step wherein multipoint mutants are exhaustively enumerated using Rosetta atomistic design calculations. In the example presented for demonstrative purposes, the PTE active site comprises a bimetal center (gray spheres) of Zn²⁺ ions that are coordinated by six highly conserved residues (gray sticks); eight additional residues (colored sticks) comprise the active-site wall and are less conserved. FIG. 1C shows the step wherein the designs are ranked by energy, and FIG. 1D shows the step wherein the sequences are clustered to obtain a repertoire of diverse, low-energy designs for experimental testing. Designed positions are colored consistently throughout FIGS. 1A-C.

As seen in FIG. 1C, each of the designed structures is subjected to a global energy minimization, based on the rules presented hereinabove, and a minimized energy scoring is determined to each of the designed structures relative to the total free energy of the template structure. According to some embodiments of the present invention, the designed structures are sorting according to the minimized energy scoring.

One of the reasons for selecting metalloenzyme phosphotriesterase (PTE) from Pseudomonas diminuta for the demonstration of the method presented herein is that in addition to highly efficient hydrolysis of the organophosphate pesticide paraoxon (k_{cat}/K_{M} approximately 10⁸ M⁻¹s⁻¹), PTE promiscuously hydrolyzes esters, lactones, and diverse organophosphates, including toxic nerve agents, such as VX, Russian VX, soman (GD), and cyclosarin (GF), albeit with k_{cat}/K_{M} values that are orders-of-magnitude lower than for paraoxon.

Effective organophosphate detoxification for in vivo protection, however, demands high catalytic efficiency, with a minimal k_{cat}/K_{M} of 10⁷ M⁻¹ min⁻¹, thereby motivating several recent enzyme-engineering efforts that targeted PTE. Furthermore, the threat from a new generation of nerve agents (“Novichoks”), similar in structure to VX and GF, reinforces the need for broad-spectrum nerve-agent hydrolases.

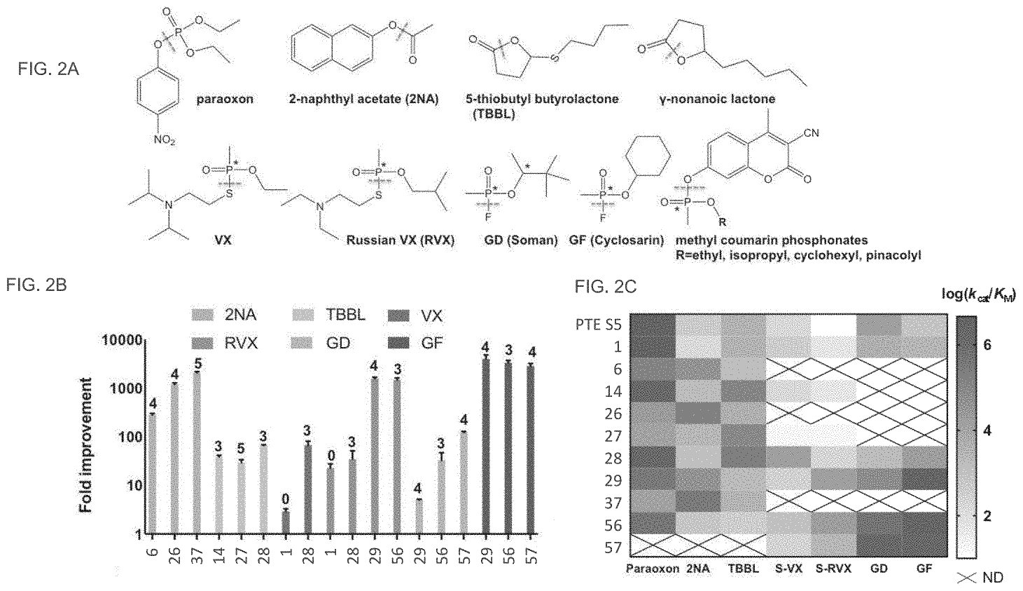

FIGS. 2A-C present some of the results of the use of the FuncLib method, according to embodiments of the present invention, in which designed repertoire of phosphotriesterases (PTE) exhibits orders of magnitude improvement in a range of promiscuous activities. Specifically, FIG. 2A shows that bacterial PTE is a paraoxonase that exhibiting additional promiscuous hydrolase activities, wherein the dashed lines indicate the bonds that PTE hydrolyses in each of the substrates tested in this study, and the asterisks indicate chiral centers. FIG. 2B shows X-fold improvement in catalytic efficiency (k_{cat}/K_{M}) of the top FuncLib designs relative to PTE-S5, showing remarkable >1,000-fold improvement in nerve-agent hydrolysis efficiency in several designs, whereas the number of active-site mutations is indicated above the bars. FIG. 2C shows the activity profiles of the top PTE designs, wherein several designs, most prominently PTE_28 (SEQ ID NO: 28), PTE_29 (SEQ ID NO: 29), and PTE_56 (SEQ ID NO: 56), exhibit substantially broadened substrate selectivity relative to the enzyme of the original sequence. Data for nerve agents are shown for the more toxic Sₚ stereoisomers. Data are represented as mean±standard deviations of duplicate measurements; N.D.—not determined. Numbers in X-axis of FIG. 2B and numbers in Y-axis in FIG. 2C represent the variant number (PTE_X) and the SEQ ID NO: X).

Since active-site mutations often impair protein stability, active-site design calculations may be started from a polypeptide chain of a stabilized design of the original polypeptide chain, namely a design provided by a method such as PROSS (see above). In the example used to demonstrate the method provided herein, the inventors employed dPTE2 (SEQ ID NO: 1), which is a variant of PTE-S5 [Roodveldt, C. and Tawfik, D. S., Protein Eng Des Sel., 2005, 18(1), pp. 51-8] with 20 stabilizing mutations outside the active-site pocket that was previously designed using the PROSS stability-design algorithm [Goldenzweig, A. et al.. Mol Cell., 2016, 63(2), pp. 337-346]. Original sequence dPTE2 (SEQ ID NO: 1) exhibited higher stability and fivefold higher bacterial-expression yields than PTE-S5, while retaining wild-type levels of activity.

Eight active-site positions that comprise the PTE active-site wall (first-shell) were selected for the design method, however, it is noted that the number of starting positions vary depending on the subject of the method and the available information thereof. The method, using FuncLib, started by defining a sequence space comprising active-site point mutations that are predicted to be individually tolerated (see, FIG. 1A). First, only mutations with at least a modest probability of occurrence in the natural diversity according to a multiple-sequence alignment of homologues were retains. Second, point mutations that substantially destabilize the original sequence (also referred to herein and throughout as “wild-type”; “starting model”; “original structure”; or “template sequence”) according to Rosetta atomistic modeling were eliminates. Applied to the PTE active-site pocket, no mutations were allowed in its Zn²⁺-chelating residues (unsubstitutable or fixed positions), whereas other first-shell positions were allowed (substitutable positions) even radical mutations (see, FIGS. 1A-B). The two-step filtering drastically reduced the combinatorial space of multipoint mutants at the eight active-site positions from 10¹⁰ mutants, if all 20 amino acids were allowed at each position, to <10⁵. From this filtered set, all the multipoint mutants that comprised 3-5 mutations relative to the original sequence were modeled and refined in Rosetta, including backbone and sidechain minimization (see, FIG. 1B). Thereafter, all multipoint mutants were ranked according to their predicted stability (see, FIG. 1C). Thus, the top-ranked designs were predicted to exhibit stable and reorganized active-site pockets—a prerequisite for high catalytic efficiency. Surprisingly, it was found that hundreds of unique active-site designs exhibited energy scores that were as favorable as or better than that of the starting sequence of PTE, suggesting that a very large space of potentially tolerated multipoint mutants at the active site was accessible by computational design. According to some embodiments, the method further includes a step wherein the designs were clustered (see, FIG. 1D), thereby eliminating designs that differed by fewer than two active-site mutations from one another or from wild-type. In this exemplary study using PTE, the top 49 designs were selected for experimental in vitro testing (see, Table 1).

Method results and sequence space:

Table 1 presents the results obtained using FuncLib as described hereinabove, starting from the original sequence of PTE, dPTE2 (SEQ ID NO: 1), and represents, at least to some extent, the sequence space of PTE variants designed for improved reactivity towards a broad spectrum of substrates. Marked in bold are the variants PTE_28 (SEQ ID NO: 28), PTE_29 (SEQ ID NO: 29), PTE_56 (SEQ ID NO: 56), and PTE_57 (SEQ ID NO: 57), which exhibited substantially broadened substrate selectivity relative to the enzyme of the original sequence.

TABLE 1

| Variant | SEQ ID | Position (numbering according to PDB entry: 1HZY | | | | | | | |
|---|---|---|---|---|---|---|---|---|---|
| (PTE_X) | NO: | 106 | 132 | 254 | 257 | 271 | 303 | 306 | 317 |
| Sequence space | | I/C/H/L/M | F/L | H/G/R | H/Y/W | L/I/R | L/T | F/I | M/L |
| per position | | | | | | | | | |
| dPTE2 | 1 | I | F | H | H | L | L | F | M |
| 2 | 2 | I | F | H | H | I | T | I | L |
| 3 | 3 | I | F | G | H | R | T | I | L |
| 4 | 4 | I | F | G | Y | L | T | I | M |
| 5 | 5 | I | F | G | Y | I | T | F | L |
| 6 | 6 | I | F | R | W | L | T | F | L |
| 7 | 7 | I | L | H | W | L | T | I | L |
| 8 | 8 | C | F | H | H | R | L | F | L |
| 9 | 9 | C | F | H | W | L | T | F | L |
| 10 | 10 | C | F | H | W | R | L | F | M |
| 11 | 11 | C | F | H | Y | I | L | F. | M |
| 12 | 12 | C | F | G | H | L | T | I | L |
| 13 | 13 | C | F | G | H | I | T | F | M |
| 14 | 14 | C | F | R | H | L | L | F | L |
| 15 | 15 | C | F | R | H | R | T | I | M |
| 16 | 16 | C | F | R | W | L | T | F | M |
| 17 | 17 | H | F | H | H | R | T | I | L |
| 18 | 18 | H | F | H | Y | L | T | I | L |
| 19 | 19 | H | F | G | H | I | L | F | M |
| 20 | 20 | H | F | G | W | I | T | F | M |
| 21 | 21 | H | F | R | H | L | T | I | L |
| 22 | 22 | H | F | R | W | L | T | I | M |
| 23 | 23 | L | F | H | H | L | T | I | L |
| 24 | 24 | L | F | H | H | R | T | F | M |
| 25 | 25 | L | F | H | W | I | L | F | L |
| 26 | 26 | L | F | H | W | I | T | F | M |
| 27 | 27 | L | F | H | Y | R | L | I | L |
| 28 | 28 | L | F | G | H | L | L | F | L |
| 29 | 29 | L | F | G | W | L | T | F | M |
| 30 | 30 | L | F | G | Y | I | T | F | M |
| 31 | 31 | L | F | R | H | I | L | I | L |
| 32 | 32 | L | F | R | H | I | T | I | M |
| 33 | 33 | L | F | R | W | R | L | F | M |
| 34 | 34 | L | F | R | Y | L | L | F | L |
| 35 | 35 | L | F | R | Y | L | L | I | M |
| 36 | 36 | L | L | H | W | L | L | F | M |
| 37 | 37 | L | L | R | W | L | T | F | M |
| 38 | 38 | M | F | H | H | L | L | I | L |
| 39 | 39 | M | F | H | H | R | T | F | L |
| 40 | 40 | M | F | H | H | R | T | I | M |
| 41 | 41 | M | F | H | W | L | T | F | M |
| 42 | 42 | M | F | H | Y | L | L | F | L |
| 43 | 43 | M | F | G | H | L | T | I | M |
| 44 | 44 | M | F | G | W | L | L | F | M |
| 45 | 45 | M | F | R | H | L | T | F | M |
| 46 | 46 | M | F | R | H | R | L | F | L |
| 47 | 47 | M | F | R | W | L | L | F | L |
| 48 | 48 | M | L | H | H | L | T | F | M |
| 49 | 49 | M | L | H | W | L | T | F | L |
| 50 | 50 | M | L | R | W | L | L | F | M |
| 51 | 51 | L | F | G | W | L | T | I | L |
| 52 | 52 | L | F | G | W | L | T | I | M |
| 53 | 53 | I | F | G | H | L | T | F | M |
| 54 | 54 | I | F | G | W | L | L | F | M |
| 55 | 55 | I | F | G | W | L | T | F | L |
| 56 | 56 | I | F | G | W | L | T | F | M |
| 57 | 57 | I | F | G | W | L | T | I | M |
| 58 | 58 | M | F | G | H | L | T | F | M |
| 59 | 59 | M | F | G | H | L | T | I | L |
| 60 | 60 | M | F | G | W | L | L | I | L |
| 61 | 61 | M | F | G | W | L | T | F | L |
| 62 | 62 | M | F | G | W | L | T | F | M |
| 63 | 63 | M | F | G | W | L | T | I | M |

RosettaScripts xml and flags files:

Refinement
refine.xml
<ROSETTASCRIPTS>

<SCOREFXNS>

<ScoreFunction name=“ref_full” weights=“ref2015”>

<Reweight scoretype=“coordinate_constraint” weight=“0.1”/>
<Reweight scoretype=“res_type_constraint” weight=“0.1”/>

</ScoreFunction>
<ScoreFunction name=“soft_rep_full” weights=“soft_rep”>

<Reweight scoretype=“coordinate_constraint” weight=“0.1”/>
<Reweight scoretype=“res_type_constraint” weight=“0.1”/>

</ScoreFunction>
<ScoreFunction name=“ref_no_pssm” weights=“ref2015”>

<Reweight scoretype=“coordinate_constraint” weight=“0.1”/>

</ScoreFunction>
<ScoreFunction name=“ref_pure” weights=“ref2015”/>

</SCOREFXNS>
<RESIDUE_SELECTORS>

<Index name=“ress_fix” resnums=“%%res_to_fix%%”/>

</RESIDUE_SELECTORS>
<TASKOPERATIONS>

<InitializeFromCommandline name=“init”/>
<RestrictToRepacking name=“rtr”/>
<OperateOnResidueSubset name=“fix_res” selector=“ress_fix”>

<PreventRepackingRLT/>

</OperateOnResidueSubset>
<OperateOnResidueSubset name=“not_to_cst_sc”>

<Not selector=“ress_fix”/>
<PreventRepackingRLT/>

</OperateOnResidueSubset>

</TASKOPERATIONS>
<MOVERS>

<AtomCoordinateCstMover name=“fix_res_sc_cst” coord_dev=“0.5” bounded=“false”

sidechain=“true” task_operations=“not_to_cst_sc”/>

<PackRotamersMover name=“soft_repack” scorefxn=“soft_rep_full”

task_operations=“init,rtr,fix_res”/>

<PackRotamersMover name=“hard_repack” scorefxn=“ref_full”

task_operations=“init,rtr,fix_res”/>

<RotamerTrialsMinMover name=“RTmin” scorefxn=“ref_full”

task_operations=“init,rtr,fix_res”/>

<TaskAwareMinMover name=“soft_min” scorefxn=“soft_rep_full” chi=“1” bb=“1”

jump=“0” task_operations=“init,fix_res”/>

<TaskAwareMinMover name=“hard_min” scorefxn=“ref_full” chi=“1” bb=“1” jump=“0”

task_operations=“init,fix_res”/>

<ConstraintSetMover name=“add_CA_cst” cst_file=“%%cst_full_path%%”/>
<ParsedProtocol name=“refinement_block”>

<Add mover_name=“soft_repack”/>
<Add mover_name=“soft_min”/>
<Add mover_name=“soft_repack”/>
<Add mover_name=“hard_min”/>
<Add mover_name=“hard_repack”/>
<Add mover_name=“hard_min”/>
<Add mover_name=“hard_repack”/>
<Add mover_name=“RTmin”/>
<Add mover_name=“RTmin”/>
<Add mover_name=“hard_min”/>

</ParsedProtocol>
<LoopOver name=“iter4” mover_name=“refinement_block” iterations=“4”/>

</MOVERS>
<FILTERS>

<ScoreType name=“stability_score_full” scorefxn=“ref_full” score_type=“total_score”

confidence=“0” threshold=“0”/>

<ScoreType name=“stability_without_pssm” scorefxn=“ref_no_pssm”

score_type=“total_score” confidence=“0” threshold=“0”/>

<ScoreType name=“stability_pure” scorefxn=“ref_pure” score_type=“total_score”

confidence=“0” threshold=“0”/>

<Rmsd name=“rmsd” confidence=“0”/>
<Time name=“timer”/>

</FILTERS>
<PROTOCOLS>

<Add filter_name=“timer”/>
<Add mover_name=“add_CA_cst”/>
<Add mover_name=“fix_res_sc_cst”/>
<Add mover_name=“iter4”/>
<Add filter_name=“stability_score_full”/>
<Add filter_name=“stability_without_pssm”/>
<Add filter_name=“stability_pure”/>
<Add filter_name=“rmsd”/>
<Add filter_name=“timer”/>

</PROTOCOLS>
<OUTPUT scorefxn=“ref_full”/>

</ROSETTASCRIPTS>

refine.flags

-use_input_sc
-extrachi_cutoff 5
-ignore_unrecognized_res
-chemical:exclude_patches LowerDNA UpperDNA Cterm_amidation SpecialRotamer VirtualBB

ShoveBB VirtualDNAPhosphate VirtualNTerm CTermConnect sc_orbitals pro_hydroxylated_case1
pro_hydroxylated_case2 ser_phosphorylated thr_phosphorylated tyr_phosphorylated tyr_sulfated
lys_dimethylated lys_monomethylated lys_trimethylated lys_acetylated glu_carboxylated cys_acetylated
tyr_diiodinated N_acetylated C_methylamidated MethylatedProteinCterm

-linmem_ig 10
-ignore_zero_occupancy false
-s # path to structure file
-out:path:pdb pdbs
-out:path:score scores
-parser:protocol refine.xml
-parser:script_vars res_to_fix= # comma separated list of positions
-parser:script_vars cst_full_path= # path to Rosetta CST file of CA atoms
Filterscan
filterscan.xml
<ROSETTASCRIPTS>

<SCOREFXNS>

<ScoreFunction name=“scorefxn_full” weights=“ref2015”>

<Reweight scoretype=“coordinate_constraint” weight=“0.1”/>
<Reweight scoretype=“res_type_constraint” weight=“0.1”/>

</ScoreFunction>

</SCOREFXNS>
<RESIDUE_SELECTORS>

<Index name=“ress_fix” resnums=“%%res_to_fix%%”/>

</RESIDUE_SELECTORS>
<TASKOPERATIONS>

<InitializeFromCommandline name=“init”/>
<DesignAround name=“des_around” design_shell=“0.1” resnums=“%%current_res%%”

repack_shell=“8.0”/>

<SeqprofConsensus name=“pssm_cutoff” filename=“%%pssm_full_path%%”

min_aa_probability=“−2” probability_larger_than_current=“0” convert_scores_to_probabilities=“0”
keep_native=“1” debug=“1” ignore_pose_profile_length_mismatch=“0”/>

<OperateOnResidueSubset name=“fix_res” selector=“ress_fix”>

<PreventRepackingRLT/>

</OperateOnResidueSubset>
<OperateOnResidueSubset name=“not_to_cst_sc”>

<Not selector=“ress_fix”/>
<PreventRepackingRLT/>

</OperateOnResidueSubset>

</TASKOPERATIONS>
<FILTERS>

<ScoreType name=“stability_score_full” scorefxn=“scorefxn_full” score_type=“total_score”

threshold=“0.0”/>

<Delta name=“delta_score_full” filter=“stability_score_full” upper=“1” lower=“0”

range=“0.5”/>

<FilterScan name=“filter_scan” scorefxn=“scorefxn_full” relax_mover=“min_all”

keep_native=“1” task_operations=“init,des_around,pssm_cutoff,fix_res” delta_filters=“delta_score_full”
delta=“true” resfile_name=“resfiles/res_%%current_res%%” report_all=“1”
delta_filter_thresholds=“0.0,0.5,1.0,1.5,2.0,2.5,3.0,3.5,4.0,4.5,5.0,5.5,6.0”
score_log_file=“scores/res%%current_res%%_score_full.log” dump_pdb=“1”/>

</FILTERS>
<MOVERS>

<AtomCoordinateCstMover name=“fix_res_sc_cst” coord_dev=“0.5” bounded=“false”

sidechain=“true” task_operations=“not_to_cst_sc”/>

<ConstraintSetMover name=“add_CA_cst” cst_file=“%%cst_full_path%%”/>
<FavorSequenceProfile name=“FSP” scaling=“none” weight=“1”

pssm=“%%pssm_full_path%%” scorefxns=“scorefxn_full” />

<MinMover name=“min_all” scorefxn=“scorefxn_full” chi=“1” bb=“1” jump=“0”/>

</MOVERS>

<PROTOCOLS>

<Add mover_name=“add_CA_cst”/>
<Add mover_name=“fix_res_sc_cst”/>
<Add mover=“FSP”/>
<Add filter=“filter_scan”/>

</PROTOCOLS>
<OUTPUT scorefxn=“scorefxn_full”/>

</ROSETTASCRIPTS>
filterscan.flags
-use_input_sc
-extrachi_cutoff 5
-ignore_unrecognized_res
-chemical:exclude_patches LowerDNA UpperDNA Cterm_amidation SpecialRotamer VirtualBB

ShoveBB VirtualDNAPhosphate VirtualNTerm CTermConnect sc_orbitals pro_hydroxylated_case1
pro_hydroxylated_case2 ser_phosphorylated thr_phosphorylated tyr_phosphorylated tyr_sulfated
lys_dimethylated lys_monomethylated lys_trimethylated lys_acetylated glu_carboxylated cys_acetylated
tyr_diiodinated N_acetylated C_methylamidated MethylatedProteinCterm

-linmem_ig 10
-ignore_zero_occupancy false
-s # path to structure file
-out:path:pdb pdbs
-out:path:score scores
-parser:protocol filterscan.xml
-parser:script_vars current_res= # a position to mutational ddG for
-parser:script_vars res_to_fix= # comma separated list of positions
-parser:script_vars cst_full_path= # path to Rosetta CST file of CA atoms
-parser:script_vars pssm_full_path= # path to pssm file
Making the designs
mutate.xml
<ROSETTASCRIPTS>

<SCOREFXNS>

<ScoreFunction name=“scorefxn_full” weights=“ref2015”>

<Reweight scoretype=“coordinate_constraint” weight=“0.1”/>

</ScoreFunction>
<ScoreFunction name=“soft_rep_full” weights=“soft_rep”>

<Reweight scoretype=“coordinate_constraint” weight=“0.1”/>
<Reweight scoretype=“res_type_constraint” weight=“0.1”/>

</ScoreFunction>

</SCOREFXNS>
<RESIDUE_SELECTORS>

<Index name=“ress_fix” resnums=“%%res_to_fix%%”/>

</RESIDUE_SELECTORS>
<TASKOPERATIONS>

<RestrictToRepacking name=“rtr”/>
<OperateOnResidueSubset name=“fix_not_neighbor”>

<Not>

Neighborhood distance=“8”>

<Index resnums=“%%all_ress%%”/>

</Neighborhood>

</Not>
<PreventRepackingRLT/>

</OperateOnResidueSubset>
<InitializeFromCommandline name=“init”/>
<IncludeCurrent name=“include_curr”/>
<OperateOnResidueSubset name=“fix_res” selector=“ress_fix”>

<PreventRepackingRLT/>

</OperateOnResidueSubset>
<OperateOnResidueSubset name=“not_to_cst_sc”>

<Not selector=“ress_fix”/>
<PreventRepackingRLT/>

</OperateOnResidueSubset>

</TASKOPERATIONS>
<MOVERS>

<MutateResidue name=“mutres0” new_res=“%%new_res0%%” target=“%%target0%%”

preserve_atom_coords=“true”/>

<MutateResidue name=“mutres1” new_res=“%%new_res1%%” target=“%%target1%%”

preserve_atom_coords=“true”/>

<MutateResidue name=“mutres2” new_res=“%%new_res2%%” target=“%%target2%%”

preserve_atom_coords=“true”/>

<MutateResidue name=“mutres3” new_res=“%%new_res3%%” target=“%%target3%%”

preserve_atom_coords=“true”/>

<MutateResidue name=“mutres4” new_res=“%%new_res4%%” target=“%%target4%%”

preserve_atom_coords=“true”/>

<MutateResidue name=“mutres5” new_res=“%%new_res5%%” target=“%%target5%%”

preserve_atom_coords=“true”/>

<MutateResidue name=“mutres6” new_res=“%%new_res6%%” target=“%%target6%%”

preserve_atom_coords=“true”/>

<MutateResidue name=“mutres7” new_res=“%%new_res7%%” target=“%%target7%%”

preserve_atom_coords=“true”/>

<ConstraintSetMover name=“add_CA_cst” cst_file=“%%cst_full_path%%”/>
<AtomCoordinateCstMover name=“fix_res_sc_cst” coord_dev=“0.5” bounded=“false”

sidechain=“true” task_operations=“not_to_cst_sc”/>

<PackRotamersMover name=“prm”

task_operations=“init,include_curr,rtr,fix_not_neighbor,fix_res” scorefxn=“scorefxn_full”/>

<RotamerTrialsMinMover name=“rtmin”

task_operations=“init,include_curr,rtr,fix_not_neighbor,fix_res” scorefxn=“scorefxn_full”/>

<MinMover name=“min” bb=“1” chi=“1” jump=“0” scorefxn=“scorefxn_full”/>
<PackRotamersMover name=“soft_repack” scorefxn=“soft_rep_full”

task_operations=“init,include_curr,rtr,fix_not_neighbor,fix_res”/>

</MOVERS>
<PROTOCOLS>

<Add mover=“add_CA_cst”/>
<Add mover=“fix_res_sc_cst”/>
<Add mover=“mutres0”/>
<Add mover=“mutres1”/>
<Add mover=“mutres2”/>
<Add mover=“mutres3”/>
<Add mover=“mutres4”/>
<Add mover=“mutres5”/>
<Add mover=“mutres6”/>
<Add mover=“mutres7”/>
<Add mover=“soft_repack”/>
<Add mover=“min”/>
<Add mover=“prm”/>
<Add mover=“min”/>

</PROTOCOLS>
<OUTPUT scorefxn=“scorefxn_full”/>

</ROSETTASCRIPTS>
mutate.flags
-use_input_sc
-extrachi_cutoff 5
-ignore_unrecognized_res
-chemical:exclude_patches LowerDNA UpperDNA Cterm_amidation SpecialRotamer VirtualBB

ShoveBB VirtualDNAPhosphate VirtualNTerm CTermConnect sc_orbitals pro_hydroxylated_case1
pro_hydroxylated_case2 ser_phosphorylated thr_phosphorylated tyr_phosphorylated tyr_sulfated
lys_dimethylated lys_monomethylated lys_trimethylated lys_acetylated glu_carboxylated cys_acetylated
tyr_diiodinated N_acetylated C_methylamidated MethylatedProteinCterm

-linmem_ig 10
-ignore_zero_occupancy false
-s # path to structure file
-parser:protocol mutate.xml
-parser:script_vars res_to_fix= # comma separated list of positions
-parser:script_vars cst_full_path= # path to Rosetta CST file of CA atoms
-parser:script_vars all_ress= # comma separated list of all library positions
Exemplary job file: job.xml
<JobDefinitionFile>
<Job>

```
<Input>
  <PDB filename=“1hzy.pdb”/>
</Input>
<Output>
  <PDB filename=“0101010101010101” path=“/dev/null” pdb_gz=“true”/>
</Output>
<Options>
  <parser_script_vars value=“target0=72A new_res0=ILE target1=98A new_res1=PHE
```

target2=220A new_res2=HIS target3=223A new_res3=HIS target4=237A new_res4=LEU target5=269A
new_res5=LEU target6=272A new_res6=PHE target7=283A new_res7=MET”/>

<out_file_scorefile value=“scores/l .sc”/>
</Options>

</Job>
</JobDefinitionFile>
Command line
rosetta_scripts_jd3.default.linuxgccrelease @mutate.flags -in:file:job_definition_file job.xml

### Example 2

### Functional Library Preparation

Materials:

Substrates were synthesized as previously published: 5-thiobutyl butyrolactone (TBBL) [Khersonsky, O. and Tawfik, D. S., Chembiochem, 2006, 7, pp. 49-53]; phosphonates with cyanocoumarin leaving group, ethyl methyl phosphocyanocoumarin (EMP), isopropyl methyl phosphocyanocoumarin (IMP), cyclohexyl methyl phosphocyanocoumarin (CMP), and pinacolyl methyl phosphocyanocoumarin (PMP) [Ashani, Y. et al., Chemico-Biological Interactions, 2010, 187(1-3), pp. 362-369]; and VX and RVX enantiomers [Berman, H. A. and Leonard, K., J. Biol. Chem., 1989, 264, pp. 3942-3950].

All the other reagents (paraoxon, malathion, p-nitrophenyl acetate, p-nitrophenyl octanoate, 2-naphthyl acetate, γ-nonanoic lactone, DTNB, m-cresol, sodium acetate, propionic acid, butyric acid, isobutyric acid, valeric acid, isovaleric acid, sodium lactate, caproic acid, NADH, lactate dehydrogenase, phosphoenol pyruvate, pyruvate kinase, adenosine 3-phosphate, coenzyme A) were purchased from Sigma-Aldrich, and yeast myokinase was purchased from Merck.

Cloning:

Synthetic genes for the original enzyme and the designed variants were codon optimized for efficient E. coli expression, and custom synthesized as linear fragments by Twist Bioscience. The genes of PTE designs were amplified and cloned into the pMal C2 vector with N-terminal MBP fusion tag through the EcoRI and PstI restriction sites. The plasmids were transformed into E. coli BL21 DE3 cells, and DNA was extracted for Sanger sequencing to validate accuracy. The plasmids with genes of active designs were deposited at AddGene (deposit number 75507).

Protein expression:

2 ml of 2YT medium supplemented with 100 μg/ml ampicillin (and 0.1 mM ZnCl₂ in case of PTE) were inoculated with a single colony and grown at 37° C. for about 15 hours. 10 ml 2YT medium supplemented with 50 μg/ml kanamycin (and 0.1 mM ZnCl₂ in case of PTE) were inoculated with 0.2 ml overnight culture and grown at 37° C. to an OD₆₀₀ of about 0.6. Overexpression was induced with 0.2 mM IPTG, and the cultures were grown for about 24 hours at 20° C. After centrifugation and storage at −20° C., the pellets were resuspended in lysis buffer and lysed by sonication.

PTE purification:

PTE lysis buffer: 50 mM Tris (pH 8.0), 100 mM NaCl, 10 mM NaHCO₃, 0.1 mM ZnCl₂, benzonase and 0.1 mg/ml lysozyme. The protein was bound to amylose resin (NEB), washed with 50 mM Tris with 100 mM NaCl and 0.1 mM ZnCl₂, and the proteins were eluted with wash buffer containing 10 mM maltose. The elution fraction was used for SDS-PAGE gel and before activity assays the proteins were dialyzed in wash buffer. For crystallization, the PTE variants were re-cloned into pETMBPH vector containing an N-terminal 6×His tag and MBP fusion [Peleg, Y. and Unger, T., Methods Mol. Biol., 2008, 426, pp. 197-208] and the expression was performed with 500 ml culture. After purification, the protein was digested with TEV protease to remove the MBP fusion tag (1:20 TEV, 1 mM DTT, 24-48 h/RT). The MBP fusion was removed by binding to Ni²⁺-NTA resin, and the protein was purified by gel filtration (HiLoad 26/600 Superdex75 preparative grade column, GE).

Kinetic measurements:

The kinetic measurements of PTE designs were performed with purified proteins in activity buffer (50 mM Tris pH 8.0 with 100 mM NaCl, and 0.1 mM ZnCl₂). A range of enzyme concentrations was used, depending on the activity. The activity of PTE designs was tested colorimetrically with phosphotriesters (paraoxon (0.5 mM), malathion (0.25 mM), EMP, IMP, CMP, PMP (0.1 mM each), esters (p-nitrophenyl acetate (0.5 mM), p-nitrophenyl octanoate (0.1 mM), 2-naphthyl acetate (0.3 mM), and lactones (TBBL) (0.5 mM), γ-nonanoic lactone (0.5 mM, pH-sensitive assay, by monitoring the absorbance of m-cresol indicator at 577 nm). The kinetic measurements were performed in 96-well plates (optical length—0.5 cm), and background hydrolysis rates were subtracted.

The rate of hydrolysis of the V-type nerve agents in presence of organophosphate (OP) hydrolases was performed as described [Cherny, I. et al., ACS Chem Biol., 2013, 8(11), pp. 2394-403]. The in situ conversion of the coumarin surrogates to the corresponding G nerve agents in diluted aqueous solutions and the monitoring of the rate of detoxification of the G agents by OP hydrolases were performed as previously described [Ashani, Y. et al., Toxicology Letters, 2011, 206, pp. 24-28; and Gupta, R. D. et al., Nat Chem Biol., 2011, 7(2), pp. 120-5]. Note that the concentration of the in situ generated G-and V-agents is non-hazardous foremost because the in situ synthesis was performed on a small (mg) scale in diluted aqueous solutions. Nonetheless, due to their high potency as inhibitors of AChE, all safety requirements were strictly observed.

Catalytic efficiencies (k_{cat}/K_{M}) were determined for the most active PTE designs by measuring the activity at several low substrate concentrations in the approximated first-order kinetics region of the Michaelis-Menten equation. All the reported values represent the averages ±standard deviations based on at least two independent measurements.

Structure determination and refinement of the PTE designs structures:

Crystals of PTE_6 (SEQ ID NO: 6), PTE_28 (SEQ ID NO: 28) and PTE_29 (SEQ ID NO: 29) were obtained using the hanging-drop vapor-diffusion method with a Mosquito robot (TTP LabTech). All data sets were collected at 100 K on a single crystal on in-house RIGAKU RU-H3R X-ray. The crystals of PTE_6 (SEQ ID NO: 6) were grown from 0.85 M Lithium sulfate and 0.05M HEPES pH=7.0. The crystals formed in the space group P4₃2₁2, with one dimer per asymmetric unit and diffracted to 1.63 Å resolution. Crystals of PTE_28 (SEQ ID NO: 28) were grown from 0.1 M MgCl₂*6H₂O, 10% PEG 4000 and 0.05 M Tris pH=7.5. The crystals formed in the space group C₂, with one dimer per asymmetric unit and diffracted to 1.9 Å resolution. Crystals of PTE_29 (SEQ ID NO: 29) were grown from 0.1 M Mg(OAC)₂*4H₂O, 8% PEG 8000 and 0.05 M Na cacodylate pH=6.4. The crystals formed in the space group C₂, with one dimer per asymmetric unit and diffracted to 1.95 Å resolution.

Diffraction images of PTE_6 (SEQ ID NO: 6), PTE_28 (SEQ ID NO: 28) and PTE_29 (SEQ ID NO: 29) crystals were indexed and integrated using the Mosflm program, and the integrated reflections were scaled using the SCALA program. Structure factor amplitudes were calculated using TRUNCATE from the CCP4 program suite. The PTE_6 (SEQ ID NO: 6), PTE_28 (SEQ ID NO: 28) and PTE_29 (SEQ ID NO: 29) structures were solved by molecular replacement with the program PHASER. The model used to solve the PTE_6 (SEQ ID NO: 6), PTE_28 (SEQ ID NO: 28) and PTE_29 (SEQ ID NO: 29) structures was the engineered organophosphorous hydrolase (PDB entry: 1QW7).

All steps of atomic refinement were carried out with the CCP4/REFMAC5 program and by Phenix refine. The models were built into 2 mF_{obs}-DF_{calc}, and mF_{obs}-DF_{calc} maps by using the COOT program. Details of the refinement statistics of the PTE_6 (SEQ ID NO: 6), PTE_28 (SEQ ID NO: 28) and PTE_29 (SEQ ID NO: 29) structures are described in Table 1. The coordinates of PTE_6 (SEQ ID NO: 6), PTE_28 (SEQ ID NO: 28) and PTE_29 (SEQ ID NO: 29) were deposited in the RCSB Protein Data Bank with accession codes 6GBJ, 6GBK and 6GBL respectively. The structures will be released upon publication.

### Example 3

### Functional Library Characterization

All PTE designs retained detectable levels of paraoxonase activity (see, Table 2 below), demonstrating that their active site was intact and functional despite the high sequence diversity.

PTE variants and paraoxon/malathion:

Table 2 presents specific activity of PTE variants (μM product/min for mg protein) with phosphotriesters paraoxon (0.5 mM) and malathion (0.25 mM).

TABLE 2

| | | Paraoxon | | | Malathion | | |
|---|---|---|---|---|---|---|---|
| | | | Specific | | | Specific | |
| Variant | SEQ ID | Specific | activity | X-fold | specific | activity, | X-fold |
| (PET_X) | NO: | activity | st. dev. | improvement | activity | st. dev. | improvement |
| dPTE2 | 1 | 1831689 | 399922 | 1 | 12.3 | 0.13 | 1 |
| 2 | 2 | 19382 | 12563 | 0.011 | ND^{a} | ND | ND |
| 3 | 3 | 24852 | 6865 | 0.0114 | 3.2 | 0.01 | 0.265 |
| 4 | 4 | 423802 | 83879 | 0.231 | 3.4 | 0.07 | 0.275 |
| 5 | 5 | 416265 | 105364 | 0.227 | 19.7 | 1.77 | 1.61 |
| 6 | 6 | 24100 | 896 | 0.013 | 5.8 | 0.45 | 0.476 |
| 7 | 7 | 4840 | 1037 | 0.003 | ND | ND | ND |
| 8 | 8 | 272243 | 18654 | 0.149 | 6.7 | 0.39 | 0.547 |
| 9 | 9 | 159772 | 9847 | 0.087 | ND | ND | ND |
| 10 | 10 | 131744 | 59833 | 0.072 | 20.6 | 2.31 | 1.683 |
| 11 | 11 | 363910 | 236417 | 0.199 | 5.5 | 0.94 | 0.448 |
| 12 | 12 | 14401 | 5901 | 0.008 | 0.9 | 0.13 | 0.070 |
| 13 | 13 | 158957 | 35117 | 0.087 | 3.1 | 0.34 | 0.256 |
| 14 | 14 | 251386 | 28715 | 0.137 | 12.4 | 1.54 | 1.008 |
| 15 | 15 | 2562 | 475 | 0.001 | 1.0 | 0.05 | 0.0081 |
| 16 | 16 | 6600 | 1163 | 0.004 | 1.4 | 0.26 | 0.117 |
| 17 | 17 | 8 | 7 | 0.000005 | ND | ND | ND |
| 18 | 18 | 60 | 42 | 0.000033 | ND | ND | ND |
| 19 | 19 | 3030 | 502 | 0.002 | ND | ND | ND |
| 20 | 20 | 330 | 22 | 0.00018 | ND | ND | ND |
| 21 | 21 | 331 | 81 | 0.00018 | ND | ND | ND |
| 22 | 22 | 8 | 1 | 0.000005 | ND | ND | ND |
| 23 | 23 | 18276 | 1338 | 0.010 | 3.2 | 0.01 | 0.26 |
| 24 | 24 | 8585 | 1463 | 0.005 | ND | ND | ND |
| 25 | 25 | 120540 | 4312 | 0.066 | 23.9 | 0.87 | 1.95 |
| 26 | 26 | 7971 | 482 | 0.004 | 4.5 | 0.50 | 0.366 |
| 27 | 27 | 7589 | 279 | 0.004 | 14.7 | 0.98 | 1.199 |
| 28 | 28 | 283534 | 27113 | 0.155 | 20.1 | 1.52 | 1.641 |
| 29 | 29 | 129516 | 38476 | 0.071 | 7.5 | 0.71 | 0.614 |
| 30 | 30 | 776019 | 105049 | 0.424 | 34.7 | 3.16 | 2.831 |
| 31 | 31 | 75590 | 1229 | 0.041 | 15.8 | 0.21 | 1.288 |
| 32 | 32 | 32664 | 9138 | 0.018 | 1.5 | 0.06 | 0.123 |
| 33 | 33 | 30701 | 1009 | 0.017 | 175.8 | 44.84 | 14.34 |
| 34 | 34 | 51106 | 8465 | 0.028 | 20.0 | 1.58 | 1.634 |
| 35 | 35 | 28392 | 9499 | 0.016 | 22.1 | 1.37 | 1.799 |
| 36 | 36 | 17941 | 510 | 0.010 | ND | ND | ND |
| 37 | 37 | 6800 | 2869 | 0.004 | 1.0 | 0.12 | 0.085 |
| 38 | 38 | 12457 | 487 | 0.007 | 0.6 | 0.02 | 0.046 |
| 39 | 39 | 272 | 139 | 0.00015 | ND | ND | ND |
| 40 | 40 | 16 | 6 | 0.00001 | ND | ND | ND |
| 41 | 41 | 1703 | 523 | 0.001 | ND | ND | ND |
| 42 | 42 | 51358 | 1581 | 0.028 | 0.5 | 0.13 | 0.037 |
| 43 | 43 | 10180 | 2911 | 0.006 | ND | ND | ND |
| 44 | 44 | 6685 | 2698 | 0.004 | 3.7 | 0.52 | 0.301 |
| 45 | 45 | 101739 | 34943 | 0.056 | ND | ND | ND |
| 46 | 46 | 14532 | 5650 | 0.008 | 3.8 | 0.37 | 0.311 |
| 47 | 47 | 5126 | 2140 | 0.003 | 1.2 | 0.08 | 0.098 |
| 48 | 48 | 10532 | 1765 | 0.006 | ND | ND | ND |
| 49 | 49 | 917 | 97 | 0.001 | ND | ND | ND |
| 50 | 50 | 2265 | 41 | 0.001 | ND | ND | ND |

The specific activities of the variants were measured with alternative, promiscuous substrates including phosphotriesters other than paraoxon, phosphonodiesters, carboxy-esters, and lactones (see, FIG. 2A). Following this initial screen, the catalytic efficiencies of the most active designs were determined. Most designs exhibited efficiency gains with respect to at least one substrate: 10 designs exhibited improved efficiencies in hydrolyzing the pesticide malathion by up to 14-fold, 15 showed similar levels of improvement (up to 16-fold) in lactonase efficiency, and 35 exhibited remarkable gains of up to 1,000-fold in esterase efficiency (see, FIGS. 2B-C, Table 3 and Table 5).

PTE variants and phosphotriesters with coumarin:

Table 3 presents specific activity of PTE variants (μM product/min for mg protein) with phosphotriesters with coumarin leaving group (0.1 mM). Bold face indicates relaxed enantioselectivity (no biphasic behavior characteristic of different hydrolysis rates of the two stereoisomers was observed).

TABLE 3

| | | EMP | | IMP | | CMP | | PMP | |
|---|---|---|---|---|---|---|---|---|---|
| | | | Specific | | Specific | | Specific | | Specific |
| Variant | SEQ ID | Specific | activity | Specific | activity | Specific | activity | Specific | activity |
| (PET_X) | NO: | activity | st. dev. | activity | st. dev | activity | st. dev. | activity | st. dev. |
| dPTE2 | 1 | 330677 | 12092 | 317718 | 4923 | 142793 | 3566 | 13943 | 1239 |
| 2 | 2 | 14010 | 587 | 2465 | 8 | 166006 | 30451 | 1558 | 39 |
| 3 | 3 | 25702 | 514 | 1779 | 71 | 12138 | 439 | 2864 | 76 |
| 4 | 4 | 92338 | 8890 | 30437 | 1899 | 17015 | 193 | 8185 | 5 |
| 5 | 5 | 28367 | 994 | 18075 | 476 | 8477 | 41 | 886 | 27 |
| 6 | 6 | 6534 | 54 | 2190 | 277 | 691 | 44 | 100 | 2 |
| 7 | 7 | 9304 | 557 | 724 | 9 | 3131 | 164 | 1549 | 72 |
| 8 | 8 | 31084 | 1763 | 20177 | 536 | 47759 | 748 | 1478 | 56 |
| 9 | 9 | 76404 | 581 | 26780 | 1015 | 18068 | 734 | 940 | 9 |
| 10 | 10 | 67124 | 1060 | 33897 | 1832 | 2344 | 221 | 1785 | 127 |
| 11 | 11 | 49016 | 1503 | 38416 | 2134 | 29633 | 34692 | 226 | 11 |
| 12 | 12 | 5751 | 20 | 1380 | 13 | 26958 | 2 | 1072 | 13 |
| 13 | 13 | 16701 | 291 | 13500 | 641 | 7211 | 20 | 1075 | 0 |
| 14 | 14 | 36002 | 266 | 27008 | 1966 | 42811 | 2289 | 159 | 7 |
| 15 | 15 | 420 | 31 | 45 | 2 | 1055 | 94 | 17 | 1 |
| 16 | 16 | 2475 | 110 | 310 | 1 | 224 | 8 | 13 | 3 |
| 17 | 17 | 16 | 1 | 3 | 0.1 | 66 | 1 | ND | ND |
| 18 | 18 | 112 | 0.01 | 23 | 1 | 149 | 9 | 5 | 0.1 |
| 19 | 19 | 5153 | 166 | 7293 | 42 | 5976 | 17 | 171 | 1 |
| 20 | 20 | 1234 | 100 | 694 | 18 | 767 | 66 | 18 | 3 |
| 21 | 21 | 37 | 2 | 15 | 0.2 | 3513 | 25 | 5 | 0.1 |
| 22 | 22 | 8 | 0.2 | 3 | 0.1 | 19 | 0.02 | ND | ND |
| 23 | 23 | 6291 | 93 | 4347 | 113 | 123657 | 12869 | 784 | 7 |
| 24 | 24 | 4822 | 97 | 4408 | 138 | 43103 | 1140 | 612 | 11 |
| 25 | 25 | 178909 | 16868 | 145402 | 8815 | 23822 | 233 | 1666 | 19 |
| 26 | 26 | 45693 | 643 | 15769 | 540 | 39817 | 149 | 329 | 9 |
| 27 | 27 | 3603 | 199 | 2749 | 59 | 10074 | 22 | 1115 | 11 |
| 28 | 28 | 136012 | 2644 | 31577 | 2726 | 2501 | 363 | 10662 | 26 |
| 29 | 29 | 69759 | 4337 | 40942 | 384 | 13061 | 94 | 2022 | 76 |
| 30 | 30 | 8951 | 1963 | 8812 | 220 | 3063 | 153 | 328 | 15 |
| 31 | 31 | 18568 | 1053 | 18288 | 20 | 155709 | 8495 | 1523 | 39 |
| 32 | 32 | 4339 | 169 | 3989 | 70 | 57811 | 2260 | 652 | 40 |
| 33 | 33 | 45044 | 3338 | 9703 | 157 | 1880 | 179 | 187 | 10 |
| 34 | 34 | 9479 | 201 | 3124 | 131 | 1260 | 38 | 95 | 4 |
| 35 | 35 | 4410 | 223 | 1005 | 36 | 360 | 17 | 13 | 1 |
| 36 | 36 | 34534 | 112 | 5548 | 110 | 402 | 15 | 137 | 4 |
| 37 | 37 | 967 | 57 | 294 | 13 | 1400 | 5 | 13 | 2 |
| 38 | 38 | 9735 | 349 | 11207 | 37 | 84039 | 9193 | 331 | 3 |
| 39 | 39 | 318 | 4 | 194 | 10 | 8489 | 325 | 48 | 1 |
| 40 | 40 | 35 | 1 | 14 | 1 | 127 | 2 | 5 | 0.2 |
| 41 | 41 | 13306 | 190 | 7461 | 244 | 4715 | 167 | 102 | 7 |
| 42 | 42 | 42443 | 494 | 23941 | 865 | 26543 | 309 | 423 | 5 |
| 43 | 43 | 4086 | 41 | 1856 | 20 | 15879 | 1119 | 437 | 13 |
| 44 | 44 | 77219 | 1393 | 31165 | 274 | 3435 | 97 | 240 | 22 |
| 45 | 45 | 5969 | 126 | 4320 | 91 | 6659 | 49 | 68 | 5 |
| 46 | 46 | 2488 | 71 | 1562 | 16 | 7348 | 175 | 68 | 6 |
| 47 | 47 | 1554 | 38 | 540 | 4 | 40 | 0.2 | 3 | 0.1 |
| 48 | 48 | 3774 | 132 | 4034 | 146 | 23786 | 313 | 93 | 17 |
| 49 | 49 | 2503 | 21 | 1375 | 14 | 3729 | 214 | 18 | 0.4 |
| 50 | 50 | 605 | 2 | 111 | 2 | 22 | 1 | 3 | 0.03 |

PTE variants and esters:

Table 4 presents specific activity of PTE variants (μM product/min for mg protein) with esters. ND=below detection limit.

TABLE 4

| | | P-nitro- | | | P-nitro- | | | | | |
|---|---|---|---|---|---|---|---|---|---|---|
| | | phenyl acetate (0.5 mM) | | | phenyl octanoate (0.1 mM) | | | Naphthyl acetate (0.3 mM) | | |
| | | | Specific | | | Specific | | | Specific | |
| Variant | SEQ ID | Specific | activity | X-fold | Specific | activity | X-fold | Specific | activity | X-fold |
| (PET_X) | NO: | activity | st. dev | improvement | activity | st. dev. | improvement | activity | st. dev. | improvement |
| dPTE2 | 1 | 94 | 7.0 | 1 | 5.0 | 0.1 | 1 | 180.1 | 0.4 | 1 |
| 2 | 2 | 239 | 24.3 | 2.55 | 60.1 | 0.6 | 11.92 | 1299.9 | 12.2 | 7.22 |
| 3 | 3 | 263 | 20.1 | 2.80 | 203.1 | 14.4 | 40.31 | 6970.3 | 724.0 | 38.72 |
| 4 | 4 | 79 | 6.8 | 0.84 | 18.2 | 0.1 | 3.61 | 139.3 | 44.9 | 0.77 |
| 5 | 5 | 101 | 17.0 | 1.07 | 8.8 | 0.1 | 1.75 | 429.1 | 66.3 | 2.38 |
| 6 | 6 | 6041 | 1042.6 | 64.27 | 17.2 | 0.0 | 3.42 | 82155.1 | 7041.5 | 456.42 |
| 7 | 7 | 536 | 47.2 | 5.70 | 241.0 | 30.0 | 47.82 | 7751.5 | 689.5 | 43.06 |
| 8 | 8 | 67 | 0.9 | 0.71 | 1.1 | 0.1 | 0.22 | 295.3 | 43.9 | 1.64 |
| 9 | 9 | 1469 | 33.0 | 15.62 | 385.1 | 56.7 | 76.41 | 11135.5 | 2549.9 | 61.86 |
| 10 | 10 | 770 | 7.0 | 8.20 | 0.9 | 0.2 | 0.18 | 1583.9 | 118.0 | 8.80 |
| 11 | 11 | 34 | 1.2 | 0.37 | ND | ND | ND | 127.1 | 24.4 | 0.71 |
| 12 | 12 | 51 | 1.6 | 0.54 | 17.7 | 0.6 | 3.52 | 57.7 | 22.7 | 0.32 |
| 13 | 13 | 60 | 0.7 | 0.64 | 77.3 | 2.8 | 15.34 | 189.3 | 52.9 | 1.05 |
| 14 | 14 | 649 | 22.5 | 6.90 | 3.9 | 0.1 | 0.78 | 1624.8 | 22.4 | 903 |
| 15 | 15 | 226 | 1.5 | 2.41 | 9.4 | 0.2 | 1.87 | 4091.4 | 1109.7 | 22.73 |
| 16 | 16 | 2197 | 275.8 | 23.37 | 1.6 | 0.1 | 0.32 | 16644.7 | 5797.5 | 92.47 |
| 17 | 17 | ND^{a} | ND | ND | 0.6 | 0.0 | 0.12 | 62.5 | 60.1 | 0.35 |
| 18 | 18 | 4 | 0.2 | 0.04 | 0.7 | 0.1 | 0.14 | 32.7 | 13.2 | 0.18 |
| 19 | 19 | ND | ND | ND | 1.1 | 0.1 | 0.21 | 7.7 | 6.7 | 0.04 |
| 20 | 20 | 4 | 0.2 | 0.04 | 1.6 | 0.2 | 0.31 | 16.0 | 8.6 | 0.09 |
| 21 | 21 | 17 | 0.4 | 0.18 | 2.9 | 0.0 | 0.57 | 120.2 | 8.2 | 0.67 |
| 22 | 22 | 19 | 0.1 | 0.20 | ND | ND | ND | 185.9 | 6.5 | 1.03 |
| 23 | 23 | 1662 | 149.6 | 17.68 | 128.1 | 3.2 | 25.42 | 1633.0 | 64.0 | 9.07 |
| 24 | 24 | 304 | 1.8 | 3.24 | 12.4 | 0.2 | 2.46 | 2053.3 | 92.9 | 11.41 |
| 25 | 25 | 8623 | 16.6 | 91.74 | 51.5 | 0.4 | 10.23 | 19146.8 | 2641.7 | 106.37 |
| 26 | 26 | 51593 | 1961.9 | 548.87 | 580.7 | 47.7 | 115.21 | 137894 | 27687 | 766.1 |
| 27 | 27 | 2689 | 364.6 | 28.61 | 28.1 | 1.9 | 5.58 | 2562.4 | 88.4 | 14.24 |
| 28 | 28 | 3243 | 33.4 | 34.50 | 123.1 | 1.6 | 24.43 | 1857.4 | 23.4 | 10.32 |
| 29 | 29 | 2575 | 58.0 | 27.40 | 206.3 | 13.4 | 40.93 | 31868.6 | 7843.9 | 177.05 |
| 30 | 30 | 1897 | 21.7 | 20.18 | 17.2 | 0.5 | 3.42 | 14487.8 | 3140.2 | 80.49 |
| 31 | 31 | 1887 | 23.9 | 20.07 | 748.6 | 38.6 | 148.52 | 11727.9 | 2369.0 | 65.16 |
| 32 | 32 | 313 | 9.6 | 3.33 | 429.7 | 1.1 | 85.27 | 17636.9 | 4869.2 | 97.98 |
| 33 | 33 | 2445 | 59.8 | 26.01 | 18.2 | 0.4 | 3.61 | 19660.3 | 527.1 | 109.22 |
| 34 | 34 | 859 | 22.2 | 9.14 | 6.9 | 0.3 | 1.36 | 7899.2 | 2119.4 | 43.88 |
| 35 | 35 | 528 | 30.7 | 5.62 | 105.4 | 15.9 | 20.92 | 375.1 | 91.9 | 2.08 |
| 36 | 36 | 2949 | 9.7 | 31.37 | 14.6 | 0.4 | 2.89 | 15538.8 | 627.5 | 86.33 |
| 37 | 37 | 100738 | 5927.9 | 1071.7 | 11.7 | 0.1 | 2.33 | 83887.1 | 6978.5 | 466.04 |
| 38 | 38 | 203 | 4.6 | 2.16 | 26.3 | 0.4 | 5.22 | 310.0 | 34.7 | 1.72 |
| 39 | 39 | 13 | 0.1 | 0.13 | 2.2 | 0.1 | 0.44 | 222.5 | 8.3 | 1.24 |
| 40 | 40 | ND | ND | ND | 1.3 | 0.0 | 0.26 | 146.6 | 7.2 | 0.81 |
| 41 | 41 | 656 | 11.3 | 6.98 | 41.1 | 3.4 | 8.16 | 2414.6 | 235.6 | 13.41 |
| 42 | 42 | 10 | 0.5 | 0.11 | ND | ND | ND | 65.3 | 18.4 | 0.36 |
| 43 | 43 | 52 | 4.7 | 0.56 | 39.1 | 0.1 | 7.75 | 152.1 | 23.4 | 0.85 |
| 44 | 44 | 52 | 2.5 | 0.55 | 3.1 | 0.1 | 0.62 | 142.6 | 2.0 | 0.79 |
| 45 | 45 | 197 | 2.9 | 2.10 | 12.4 | 0.5 | 2.45 | 1270.8 | 153.7 | 7.06 |
| 46 | 46 | 128 | 4.3 | 1.36 | ND | ND | ND | 1605.7 | 21.8 | 8.92 |
| 47 | 47 | 67 | 0.2 | 0.71 | 3.1 | 0.3 | 0.61 | 164.1 | 1.2 | 0.91 |
| 48 | 48 | 101 | 2.4 | 1.08 | 9.4 | 0.1 | 1.86 | 1224.6 | 156.7 | 6.80 |
| 49 | 49 | 552 | 37.9 | 5.87 | 158.9 | 7.4 | 31.52 | 3774.7 | 283.7 | 20.97 |
| 50 | 50 | 78 | 2.6 | 0.83 | 5.1 | 0.2 | 1.01 | 110.2 | 22.2 | 0.61 |

PTE variants and lactones:

Table 5 presents specific activity of PTE variants (μM product/min for mg protein) with lactones. ND=below detection limit.

TABLE 5

| | | TBBL (0.5 mM) | | | γ-Nonanoic lactone (0.5 mM) | | |
|---|---|---|---|---|---|---|---|
| | | | Specific | | | Specific | |
| Variant | SEQ ID | Specific | activity | X-fold | Specific | activity | X-fold |
| (PET_X) | NO: | activity | st. dev. | improvement | activity | st. dev. | improvement |
| dPTE2 | 1 | 3016 | 497.9 | 1 | 126.6 | 1.35 | 1 |
| 2 | 2 | 389 | 160.8 | 0.13 | ND | | |
| 3 | 3 | 69 | 16.2 | 0.02 | ND | | |
| 4 | 4 | 134 | 49.9 | 0.04 | 368.2 | 105.0 | 2.91 |
| 5 | 5 | 200 | 116.5 | 0.07 | ND | | |
| 6 | 6 | 112 | 1.3 | 0.04 | ND | | |
| 7 | 7 | 31 | 8.5 | 0.01 | ND | | |
| 8 | 8 | 6847 | 1549.6 | 2.27 | 276.0 | 97 | 2.18 |
| 9 | 9 | 21 | 0.1 | 0.01 | ND | | |
| 10 | 10 | 5426 | 1325.2 | 1.80 | ND | | |
| 11 | 11 | 5871 | 3171.8 | 1.95 | ND | | |
| 12 | 12 | 32 | 19.2 | 0.01 | ND | | |
| 13 | 13 | 56 | 7.1 | 0.02 | ND | | |
| 14 | 14 | 14438 | 3271.7 | 4.79 | 854.3 | 7.3 | 6.75 |
| 15 | 15 | 1340 | 532.3 | 0.44 | ND | | |
| 16 | 16 | 157 | 69.5 | 0.05 | ND | | |
| 17 | 17 | 32 | 1.6 | 0.01 | ND | | |
| 18 | 18 | 82 | 27.6 | 0.03 | ND | | |
| 19 | 19 | 80 | 19.1 | 0.03 | ND | | |
| 20 | 20 | 15 | 5.9 | 0.01 | ND | | |
| 21 | 21 | 1100 | 244.6 | 0.36 | 126.0 | | 0.99 |
| 22 | 22 | 128 | 6.7 | 0.04 | ND | | |
| 23 | 23 | 538 | 87.3 | 0.18 | ND | | |
| 24 | 24 | 1825 | 107.9 | 0.61 | ND | | |
| 25 | 25 | 15299 | 168.9 | 5.07 | ND | | |
| 26 | 26 | 912 | 279.1 | 0.30 | ND | | |
| 27 | 27 | 20173 | 501.7 | 6.69 | 184.3 | 41.8 | 1.456 |
| 28 | 28 | 8739 | 296.2 | 2.90 | 1570.3 | 391.3 | 12.40 |
| 29 | 29 | 360 | 51.0 | 0.12 | ND | | |
| 30 | 30 | 4471 | 1804.8 | 1.48 | 402.2 | 174.1 | 3.18 |
| 31 | 31 | 10243 | 2150.1 | 3.40 | 2923.3 | 574.2 | 23.09 |
| 32 | 32 | 2068 | 38.6 | 0.69 | 375.9 | 16.7 | 2.99 |
| 33 | 33 | 20622 | 3688.8 | 6.84 | 7022.1 | 1065.5 | 55.47 |
| 34 | 34 | 12126 | 155.5 | 4.02 | 854.9 | 294.9 | 6.75 |
| 35 | 35 | 8988 | 1767.6 | 2.98 | 1196.9 | 413.7 | 9.45 |
| 36 | 36 | 443 | 141.4 | 0.15 | ND | | |
| 37 | 37 | 1240 | 143.5 | 0.41 | ND | | |
| 38 | 38 | 3933 | 1040.5 | 1.30 | 322.6 | 41.0 | 2.55 |
| 39 | 39 | 196 | 108.9 | 0.07 | ND | | |
| 40 | 40 | 38 | 17.1 | 0.01 | ND | | |
| 41 | 41 | 18 | 5.1 | 0.01 | ND | | |
| 42 | 42 | 985 | 11.0 | 0.33 | ND | | |
| 43 | 43 | 920 | 193.8 | 0.31 | ND | | |
| 44 | 44 | 342 | 244.4 | 0.11 | ND | | |
| 45 | 45 | 467 | 75.1 | 0.15 | 130.9 | | 1.03 |
| 46 | 46 | 4101 | 1261.2 | 1.36 | 2646.4 | 126.5 | 20.90 |
| 47 | 47 | 675 | 251.3 | 0.22 | ND | | |
| 48 | 48 | 80 | 33.1 | 0.03 | ND | | |
| 49 | 49 | 12 | 3.1 | 0.004 | ND | | |
| 50 | 50 | 683 | 265.1 | 0.23 | ND | | |

In addition to exhibiting improved catalytic efficiencies against a range of substrates, the PTE variants presented herein, according to some embodiments of the present invention, also showed vast changes in substrate selectivity. For instance, PTE-S5 is selective for paraoxon over the ester 2-naphthyl acetate (2NA) by 3×10⁴-fold. Through only five active-site mutations, selectivity has been reversed in the variant PTE_37 (SEQ ID NO: 37) to 0.04; a nearly million-fold selectivity switch. Similarly, PTE-S5 favors paraoxon over the synthetic lactone tetrabutyl butyrolactone (TBBL) by 10³-fold, whereas in design PTE_27 (SEQ ID NO: 27) selectivity is switched to 0.1 (see, Table 6 below).

Catalytic efficiency of PTE variants:

Table 6 presents specificity changes (as ratios of catalytic efficiency, k_{cat}/K_{M}) in PTE variants.

TABLE 6

| | | | Specificity | | |
|---|---|---|---|---|---|
| | | Paraoxon/ | switch | | Specificity |
| Variant | SEQ ID | 2-naphthyl | relative to | Paraoxon/ | switch relative |
| (PET_X) | NO: | acetate | dPTE2 | TBBL | to dPTE2 |
| dPTE2 | 1 | 31048.6 | 1 | 1406.5 | 1 |
| 6 | 6 | 3.41 | 9104 | 98.7 | 14 |
| 14 | 14 | 1149.3 | 27 | 15.7 | 90 |
| 25 | 25 | 25.65 | 1210 | 7.6 | 186 |
| 26 | 26 | 0.13 | 246732 | 5.2 | 272 |
| 27 | 27 | 4.61 | 6737 | 0.1 | 11219 |
| 28 | 28 | 1454.3 | 21 | 8.8 | 161 |
| 29 | 29 | 7.60 | 4086 | 148.0 | 10 |
| 37 | 37 | 0.04 | 741664 | 4.1 | 347 |
| 54 | 54 | 591 | 53 | 1206.5 | 1 |

Remarkably, these designs retained substantial paraoxonase activity (k_{cat}/K_{M}≥10⁴ M⁻¹s⁻¹), demonstrating that some of the designs broadened substrate recognition rather than only trading off one activity for another (see, FIG. 2C). Consistent with this conclusion, several designs exhibited increased efficiency with respect to the disfavored stereoisomer of methyl coumarin phosphonates relative to the wild type, while retaining high efficiency against the natively favored stereoisomer (see, Table 3).

Next, the catalytic efficiency of the designs that retained high phosphotriesterase activity with the toxic nerve agents VX, Russian VX (RVX), Soman (GD), and Cyclosarin (GF) was measured (see, Table 7 and Table 8).

Table 7 presents activity of PTE variants with nerve agents of V type, k_{cat}/K_{M} s-1M-1.

TABLE 7

| Variant | | VX | | RVX | |
|---|---|---|---|---|---|
| (PTE_X) | SEQ ID NO: | S-isomer | R-isomer | S-isomer | R-isomer |
| PTE S5 | — | 157 ± 12 | 113 ± 3 | 10.0 ± 1.6 | 333 ± 22 |
| dPTE2 | 1 | 317 ± 67 | 400 ± 12 | 217 ± 67 | 1833 ± 167 |
| 4 | 4 | 141.7 | 40 | 1650 | <16 |
| 5 | 5 | 250.0 | 110 | 1567 | <16 |
| 8 | 8 | <16 | 30 | 18 | <16 |
| 10 | 10 | 35 | 183 | 23 | <16 |
| 11 | 11 | 60 | 72 | 18 | <16 |
| 14 | 14 | 152 ± 1 | 62 | 50 | 500 |
| 25 | 25 | 116 ± 10 | 650 ± 47 | 100 | NM |
| 27 | 27 | <16 | 18 | <16 | <16 |
| 28 | 28 | 11,000 ± 2333 | 4000 ± 167 | 333 ± 166 | 11,500 ± 1000 |
| 29 | 29 | 700 ± 50 | <25 | 15,500 ± 1167 | <25 |
| 30 | 30 | 666 ± 166 | 333 ± 166 | 5500 ± 500 | 210 |
| 31 | 31 | 33 | | 27 | 122 |
| 33 | 33 | <16 | 133 | <16 | <16 |
| 34 | 34 | <16 | | <16 | <16 |
| 35 | 35 | <16 | | <16 | <16 |
| 51 | 51 | 35 | | 283 | <33 |
| 52 | 52 | 750 | | 1133 | <33 |
| 53 | 53 | 917 | | 7500 | 833 |
| 54 | 54 | 4833 | | 467 | <33 |
| 55 | 55 | 483 | | 8167 | <33 |
| 56 | 56 | 717 ± 100 | <25 | 14670 ± 1500 | <25 |
| 57 | 57 | 250 ± 50 | <25 | 2667 ± 117 | <33 |
| 58 | 58 | 138 | | 3000 | <33 |
| 59 | 59 | 20 | | 300 | <33 |
| 60 | 60 | 45 | | 67 | <33 |
| 61 | 61 | 80 | | 2667 | <33 |
| 62 | 62 | 90 | | 8167 | <33 |
| 63 | 63 | 40 | | 900 | <33 |

Table 8 presents comparison of best PTE designs activity with nerve agents with that of PTE variants obtained by directed evolution; k_{cat}/K_{M},×10⁶ M⁻¹min⁻¹, measured in 50 mM Tris with 50 mM NaCl at pH 8, 25° C.

TABLE 8

| | SEQ | | | | |
|---|---|---|---|---|---|
| | ID | | | | |
| Variant | NO: | GF | GD | S-VX | S-RVX |
| PTE-S5^{a} | — | 0.048 ± | 0.98 ± 0.31 | 0.0094^{a} | 0.0006^{a} |
| | | 0.008^{a} | (0.11 ± | 0.01^{c} | 0.0009^{c} |
| | | 0.124 ± | 0.03)^{a, b} | | |
| | | 0.009^{c} | 0.099 ± | | |
| | | | 0.005^{c} | | |
| dPTE2 | 1 | 0.170 ± | 0.29 ± 0.06 | 0.019 ± 0.004 | 0.013 ± 0.004 |
| | | 0.003 | (0.10 ± 0.01) | | |
| PTE_28 | 28 | 1.06 ± 0.11 | 0.11 ± 0.017 | 0.66 ± 0.14 | 0.02 ± 0.01 |
| PTE_29 | 29 | 191 ± 36 | 3.9 ± 0.2 | 0.042 ± 0.003 | 0.93 ± 0.07 |
| PTE_56 | 56 | 159 ± 19 | 31.2 ± 14.0 | 0.043 ± 0.006 | 0.88 ± 0.09 |
| | | | (6.2 ± 1.2) | | |
| PTE_57 | 57 | 136 ± 18 | 119.5 ± 4.9 | 0.015 ± 0.003 | 0.16 ± 0.7 |
| | | | (20.5 ± 13.4) | | |
| C23^{c} | | 1.74 ± 0.23 | 2.64 ± 0.16 | 5.95 ± 0.16 | 0.45 ± 0.01 |
| IV-A1^{c} | | 1.86 ± 0.18 | 1.53 ± 0.05 | 2.53 ± 0.11 | 5.27 ± 0.16 |
| d1- | | | 3.8 | 3.5 | 12 |
| IVA1^{d} | | | (1.1)^{b} | | |
| PROSS | | | | | |
| stabilized | | | | | |
| 10-2- | | | 1.4 | 50 | 3.2 |
| C3^{d} | | | (0.2)^{b} | | |
| stabilized | | | | | |

^{a}Data for wt-PTE-S5 taken from Cherny et al. [Cherny, I. et al., ACS Chem Biol., 2013, 8(11), pp. 2394-403]. Determined at 25° C., by use of both the DTNB and the loss of anti-AChE protocols.
^{b}In some cases, detoxification of the two S-enantiomers of GD was biphasic, which is attributed to the two toxic isomers, SₚC_{R} and S_{P}C_{S}. The parameters for the slow phase are given in the parentheses.
^{c}Data from Goldsmith et al. [Goldsmith, M. et al., Arch. Toxicol., 2016, 90, pp. 2711-2724.]. All entries determined with authentic nerve agents at 37° C. using the protocol of monitoring the ani-AChE loss of the OPs.
^{d}Data from Goldsmith et al. [Goldsmith, M. and Tawfik, D. S., Curr. Opin. Struct. Biol., 2017, 47, pp. 140-150].

As can be seen in Table 8, PTE_28 (SEQ ID NO: 28) exhibited 66-fold increase in VX hydrolysis efficiency relative to wild-type PTE, and PTE_29 (SEQ ID NO: 29) exhibited remarkable gains in efficiency of 1,550 and 3,980-fold in hydrolyzing RVX and GF, respectively.

Starting from PTE_28 (SEQ ID NO: 28), a second round of design was initiated, this time directing FuncLib to model all combinations of 3-5 mutations that occurred in the best nerve-agent hydrolases tested in the first round and eliminating designs that were predicted to be unstable (>8 Rosetta energy units relative to PTE_28 (SEQ ID NO: 28)). The 14 resulting designs were experimentally tested, finding that designs PTE_56 (SEQ ID NO: 56) and PTE_57 (SEQ ID NO: 57) exhibited increased activities towards GD (32-fold and 122-fold, respectively), and both designs exhibited a 3,000-fold increase in hydrolyzing GF. These variants, with k_{cat}/k_{M}≥10⁷ M⁻¹min⁻¹ for the highly toxic nerve agents RVX, GD, and GF, may be suitable for in vivo detoxification.

As can further be seen in Table 8, the efficiency gains observed by testing 63 variants were comparable to the best variants from the application of more than a dozen rounds of diversification and experimental testing of thousands of variants using conventional laboratory-evolution strategies. Furthermore, laboratory-evolution experiments demand separate selection campaigns for each substrate, whereas the designed repertoire comprised dozens of enzymes with improved efficiency towards each of the substrates we tested. Additionally, all of the variants showed bacterial-expression levels comparable to the highly expressed dPTE2 (SEQ ID NO: 1) starting sequence (>300 mg protein per liter culture).

These results demonstrate that the combination of PROSS and FuncLib may not exhibit the stability-threshold bottlenecks that have constrained the laboratory evolution of many enzymes, including PTE. Thus, FuncLib results in a small but functionally highly diverse repertoire of stable and efficient enzymes and may in some cases bypass the requirement for high-throughput screens.

Sequence space for PTE:

Table B presents the sequence space of amino acid substitutions (mutations) resulting from the method presented herein (FuncLib), imposing the key residues described above and allowing active-site residues to be substituted. The sequence space has 8 amino acid substitution positions, each with at least one optional substitution over the WT (or starting sequence) amino acid at the given position, wherein the original (wild type) amino acid in the position is marked by bold face and is the first from the left.

TABLE B
Position (numbering according to PDB entry: 1HZY

| 106 | 132 | 254 | 257 | 271 | 303 | 306 | 317 |
|---|---|---|---|---|---|---|---|
| I/C/H/L/M | F/L | H/G/R | H/Y/W | L/I/R | L/T | F/I | M/L |

### Example 4

### Structural Bases of Catalytic Efficiency and Selectivity

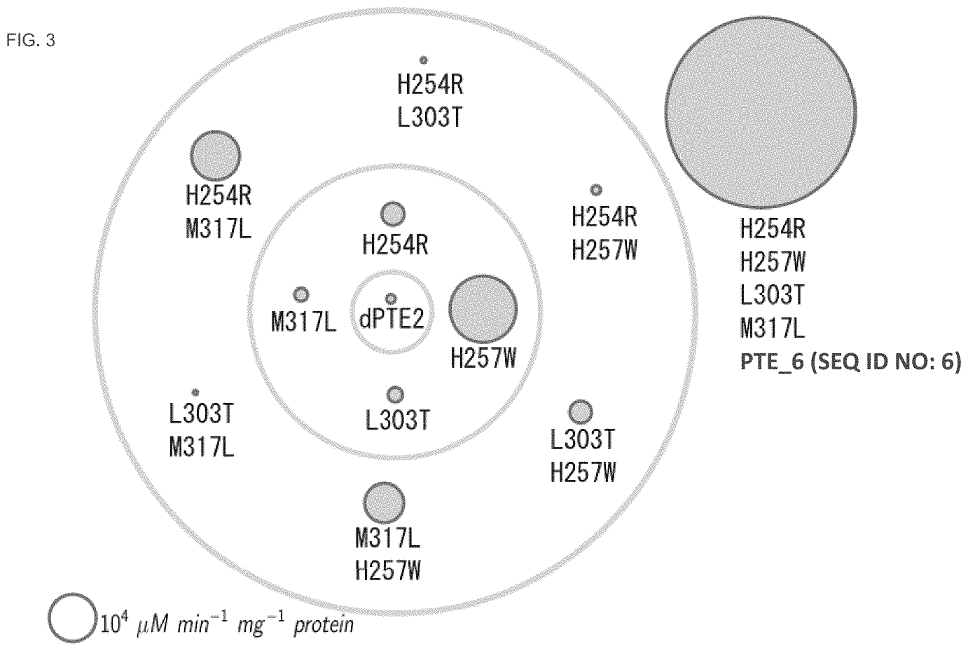

To understand what molecular factors underlie the high gains in catalytic efficiency in some variants obtained by implementing the design method provided herein, X-ray crystallography was used to determine the molecular structures of PTE_6 (SEQ ID NO: 6) (280-fold improved activity with 2NA), PTE_28 (SEQ ID NO: 28) (65-fold improved activity with TBBL and 103-fold improved activity with S-VX), and PTE_29 (SEQ ID NO: 29) (3,980-fold improved activity with GF), and the results are presented in FIG. 3 and Table 9.

FIG. 3 presents a diagram showing that the designed mutations in the PTE variants provided herein, according to some embodiments of the present invention, exhibit sign-epistatic relationships, wherein each circle represents a mutant of dPTE2 (SEQ ID NO: 1), the area of each circle is proportional to the variant's specific activity in hydrolyzing the aryl ester 2-naphthyl acetate (2NA), and wherein the PROSS designed and stabilized sequence dPTE2 (SEQ ID NO: 1), which was used as the starting point in the method provided herein, exhibits low specific activity, and each of the point mutants exhibits improved specific activity, the specific activity declines in the double mutants, and the quad-mutant, design PTE_6 (SEQ ID NO: 6), substantially improves specific activity relative to all single or double mutants.

Table 9 presents crystallographic data collection and refinement statistics for the PTE designs, wherein values in parentheses refer to the data of the corresponding upper resolution shell.

TABLE 9

| | PTE_6 | PTE_28 | PTE_29 |
|---|---|---|---|
| Variant | (SEQ ID NO: 6) | (SEQ ID NO: 28) | (SEQ ID NO: 29) |
| PDB Entry ID | 6GBJ | 6GBK | 6GBL |
| Space group | P4₃2₁2 | C2 | C2 |
| Cell dimensions: | | | |
| a, b, c (Å) | 69.49, 69.49, 186.02 | 156.75, 53.09, 89.23 | 55.80, 53.56, 89.34 |
| α, β, γ (°) | 90, 90, 90 | 90, 106.81, 90 | 90, 107.21, 90 |
| No. of copies in a.u. | 1 | 1 | 1 |
| Resolution (Å) | 38.65-1.63 | 41.47-1.9 | 41.61-1.95 |
| Upper resolution shell | 1.69-1.63 | 1.97-1.9 | 2.02-1.95 |
| (Å) | | | |
| Unique reflections | 57,720 (5,611) | 55,705 (5,523) | 45,387 (3,967) |
| Completeness (%) | 99.70 (98.79) | 99.91 (99.87) | 87.83 (77.54) |
| Multiplicity | 7.4 (7.3) | 3.3 (3.2) | 7.4 (7.3) |
| Average I/σ(I) | 13.5 (2.8) | 5.56 (1.49) | 10.91 (3.05) |
| Rsym (I) (%) | 0.0338 (0.262) | 0.09026 (0.4785) | 0.0456 (0.224) |
| Refinement: | | | |
| Resolution range (Å) | 38.65-1.63 | 41.47-1.9 | 41.61-1.95 |
| No. of reflections | 57,716 | 55,668 | 45,382 |
| (I/σ(I) > 0) | | | |
| No. of reflections in | 2,886 | 2,783 | 2,272 |
| test set | | | |
| R-working (%)/R- | 0.1696/0.1891 | 0.2010/0.2182 | 0.1833/0.2253 |
| free (%) | | | |
| No. of protein atoms | 2,558 | 5,064 | 5063 |
| No. of water | 330 | 659 | 660 |
| molecules | | | |
| Overall average B | 18.54 | 11.32 | 18.61 |
| factor (Å²) | | | |
| Root mean square | | | |
| deviations: | | | |
| bond length (Å) | 0.025 | 0.011 | 0.018 |
| bond angle (°) | 2.36 | 1.53 | 1.85 |
| Ramachandran Plot: | | | |
| Most favored (%) | 96.95 | 96.47 | 96.31 |
| Additionally allowed | 3.05 | 3.53 | 3.69 |
| (%) | | | |
| Disallowed (%) | 0.0 | 0.0 | 0.0 |

Structural insights:

Visual inspection and position analysis of the crystal structures revealed that all three structures showed high accuracy relative to their respective models (root mean square deviation [rmsd] <0.5 Å over the backbone and 0.3 Å all-atom RMSD in mutated active-site residues), confirming that the design process resulted in precise and preorganized active-site pockets as required for high-efficiency catalysis.

The crystal structures were also compared to the structures obtained in molecular docking simulations, which were generated to model the toxic Sₚ stereoisomers of VX, RVX, and GD in the active-site pockets of PTE_28 (SEQ ID NO: 28), PTE_29 (SEQ ID NO: 29), and PTE_56 (SEQ ID NO: 56), respectively. The resulting models indicated that the designed active-site pockets were large enough to accommodate the bulky nerve agents and form direct contacts with them, mostly due to two large-to-small substitutions, His254Gly and Leu303Thr (see, FIG. 3). These direct contacts may also underlie the high enantioselectivity observed in some designs (>10⁴ for design PTE_29 (SEQ ID NO: 29); see. Table 7). Furthermore, several improved esterases and lactonases (PTE_14-16 (SEQ ID NOs: 14-16), 31-35 (SEQ ID NOs: 31-35), and 37 (SEQ ID NO: 37)) encoded the His254Arg mutation, which changed the steric and electrostatic organization of the active-site pocket, as also reported in laboratory-evolution studies that enhanced these activities. It is therefore concluded that the FuncLib-designed mutations mostly affected the structure of the active-site pocket, that the designed repertoire encoded substantial stereochemical diversity in the active site leading to large selectivity changes, and that a handful of active-site mutations was sufficient to effect orders-of-magnitude improvements in catalytic efficiency and selectivity against several substrates.

Sign epistasis among designed mutations:

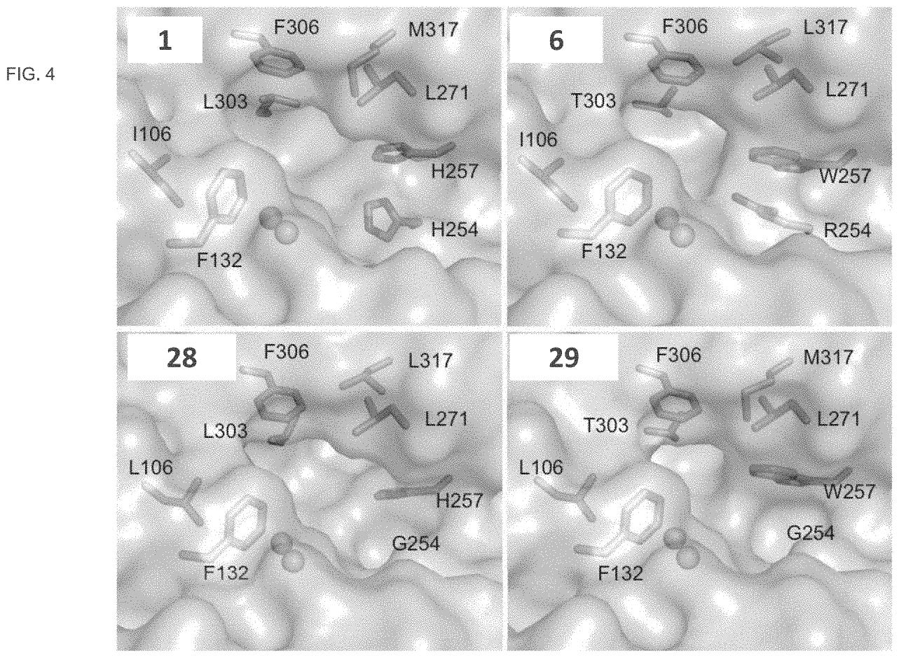

In each variant of PTE, according to some embodiments of the present invention, the mutations are spatially clustered. It was therefore anticipated that some designs would show complex epistatic relationships, whereby the effects of multipoint mutants could not be simply predicted based on the effects of the single-point mutants. The specific activities of all single- and double-point mutants comprising three of the best designs were therefore measured: PTE_6 (SEQ ID NO: 6), PTE_28 (SEQ ID NO: 28), and PTE_33 (SEQ ID NO: 33) with four, three, and four active-site mutations relative to PTE, respectively (see, FIG. 4). In PTE_6 (SEQ ID NO: 6) and PTE_33 (SEQ ID NO: 33), the point mutations improved catalytic efficiency relative to the wild type, but some double mutants exhibited efficiencies that were substantially lower than those of the wild type.

FIG. 4 presents an illustration of the stereochemical properties of the designed active-site pockets underlie selectivity changes in PTE variants, provided herein according to some embodiments of the present invention, wherein PTE_28 (SEQ ID NO: 28; denoted 28 in FIG. 4) and PTE_29 (SEQ ID NO: 29; denoted 29 in FIG. 4) exhibit a larger active-site pocket than dPTE2 (SEQ ID NO: 1; denoted 1 in FIG. 4) and high catalytic efficiency against bulky V- and G-type nerve agents (in clockwise order from top-left, molecular renderings are based on PDB entries: 1HZY, 6GBJ, 6GBK, and 6GBL; spheres indicate ions of the bimetal center.

As can be seen in FIG. 4, PTE_6 (SEQ ID NO: 6; denoted 6 in FIG. 4) provided a compelling case of sign epistasis, wherein all point mutations improved specific activity with the ester 2NA. All double mutants, however, were worse than the single-point His257Trp, and three of the double mutants were even worse than the starting point dPTE2 (SEQ ID NO: 1; denoted 1 in FIG. 4). Most revealing, the combination of two double mutants that exhibited lower specific activities than dPTE2 (SEQ ID NO: 1; denoted 1 in FIG. 4), His254Arg/His257Trp and Leu303Thr/Met317Leu, resulted in the most active design PTE_6 (SEQ ID NO: 6; denoted 6 in FIG. 4), which improved specific activity by two orders of magnitude relative to dPTE2 (SEQ ID NO: 1; denoted 1 in FIG. 4) and by three orders of magnitude relative to the Leu303Thr/Met317Leu double mutant. Furthermore, at the level of DNA, the point mutations His→Trp and Leu→Thr require three and two nucleotide exchanges, respectively, drastically reducing the odds for the emergence of PTE_6 (SEQ ID NO: 6; denoted 6 in FIG. 4) through stepwise accumulation of mutations. A previous analysis of mutational trajectories leading to enhanced fitness in clinically isolated β-lactamase mutants noted the pervasiveness of sign epistasis in evolution; and yet, a fraction of the trajectories in that case showed monotonous, and therefore evolutionarily selectable, improvement in activity. For PTE_6 (SEQ ID NO: 6; denoted 6 in FIG. 4), by contrast, the currently presented analysis suggested not even a single mutational trajectory of monotonously increasing activity. Hence, the method provided herein (FuncLib) may access mutants that cannot be obtained through the stepwise accumulation of beneficial mutations that is a prerequisite for natural or laboratory evolution.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1-8. (canceled)

9. A method for designing a plurality of non-naturally occurring polypeptide variants having an augmented activity compared to an activity of an original polypeptide, comprising:
A: providing a protein expression vector for a protein expression system, said vector comprises protein sequence data obtained by computationally designing the variants starting from said original polypeptide chain, wherein said computationally designing comprises the steps of:
(i) providing a template structure that is structurally homologous to the structure of the original polypeptide and optionally subjecting said template structure to weighted fitting energy minimization;
(ii) providing a plurality of polypeptide sequences that are each homologous to the amino-acid sequence of the original polypeptide;
(iii) defining a first shell comprising residues at a distance of 5-8 Å around residues of an active/binding site in said template structure;
(iv) within said first shell identifying substitutable positions and optionally identifying unsubstitutable positions in the amino-acid sequence of the original polypeptide;
(v) simultaneously permuting at least 2 mutations of said substitutable residues according to a PSSM threshold and a ΔΔG threshold, thereby obtaining a list of variants; and
(vi) enumerating and subjecting each of the variants to weighted fitting energy minimization, and ranking the variants by a stability score, and
B: cloning and expressing the variants in said protein expression system using said protein expression vector.

10. The method of claim 9, wherein step (vi) further comprises ranking the variants by ligand-binding affinity score.

11. The method of claim 9, further comprising, subsequent to step (vi):
(vii) selecting a subset of the variants according to said stability score.

12. The method of claim 9, further comprising, subsequent to step (vii):
(viii) filtering redundant sequences in said by clustering into representative sequences.

13. The method of claim 9, wherein said PSSM threshold is >-2 R.e.u, and said ΔΔG threshold is ≤+6 R.e.u.

14. The method of claim 9, wherein said template structure is a stabilized variant of the original polypeptide.

15. The method of claim 9, wherein step (i) comprises subjecting said template structure to weighted fitting energy minimization.

16. The method of claim 9, wherein step (i) comprises threading the amino-acid sequence of the original polypeptide on a structure of a polypeptide having at least 30% sequence identity with respect to the original polypeptide, and subjecting the threaded structure to weighted fitting energy minimization.

17. The method of claim 9, wherein said energy minimization comprises iterations of rotamer sampling followed by side chain and backbone energy minimization.

18. The method of claim 9, further comprising, prior to step (v), defining a second shell comprising residues at a distance of 5-8 Å around residues of said first shell, and within said second shell identifying additional substitutable positions in the amino-acid sequence of the original polypeptide.

19. A variant having a sequence selected from the group consisting of any combination of at least 2 amino acid substitutions of a sequence space presented in Table A, afforded using the method of claim 9 and phosphotriesterase (PTE) Pseudomonas diminuta as the original polypeptide:
TABLE A
Position (numbering according to PDB entry: 1HZY

| 106 | 132 | 254 | 257 | 271 | 303 | 306 | 317 |
|---|---|---|---|---|---|---|---|
| I/C/H/L/M | F/L | H/G/R | H/Y/W | L/I/R | L/T | F/I | M/L |

20. The variant of claim 19, being a hybrid protein wherein said combination of amino acid substitutions is implemented on a PTE protein other than said original protein.

21. The variant of claim 20, having a sequence selected from the group consisting of presented in Table 1 set forth hereinabove.

22. The variant of claim 20, having a sequence selected from the group consisting of PTE_28 (SEQ ID NO: 28), PTE_29 (SEQ ID NO: 29), PTE_56 (SEQ ID NO: 56), and PTE_57 (SEQ ID NO: 57).
